(19) United States
(12) Patent Application Publication
(10) Pub. No.: US 2024/0251792 A1
(43) Pub. Date: Aug. 1, 2024

(54) WEED CONTROL METHOD

(71) Applicant: SYNGENTA CROP PROTECTION AG, Basel (CH)

(72) Inventors: William Guy WHITTINGHAM, Bracknell, Berkshire (GB); James Nicholas SCUTT, Bracknell, Berkshire (GB); Alan Joseph HENNESSY, Bracknell, Berkshire (GB); Elizabeth Catherine FRYE, Bracknell, Berkshire (GB); Suzanna Jane DALE, Bracknell, Berkshire (GB); Ian Henry ASPINALL, Bracknell, Berkshire (GB)

(73) Assignee: SYNGENTA CROP PROTECTION AG, Basel (CH)

(21) Appl. No.: 18/561,997

(22) PCT Filed: May 12, 2022
(86) PCT No.: PCT/EP2022/062914

§ 371 Date: Nov. 17, 2023

(30) Foreign Application Priority Data
May 19, 2021 (EP) 21174854.6

(51) Int. Cl. A01N 37/10; A01P 13/00; C07C 57/30

(52) U.S. Cl. CPC A01N 37/10; A01P 13/00; C07C 57/30

(57) ABSTRACT

The present invention relates to the use of a compound of Formula (I), wherein R1, R2, R3, R4, R5 and R6 are as defined herein or an agronomically acceptable salt of said compound as a herbicide. The invention further relates to herbicidal compositions which comprise a compound of Formula (I) and to the use of compounds of Formula (I) for controlling weeds, in particular in crops of useful plants.

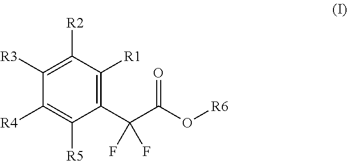

The present invention relates to the use of certain compounds as herbicides, to herbicidal compositions which comprise the compounds, and to their use for controlling weeds, in particular in crops of useful plants, or for inhibiting plant growth.

The present invention is based on the finding that certain difluoro phenylacetic acids of formula (I) as defined herein, exhibit surprisingly good herbicidal activity. Thus, according to the present invention there is provided the use of a compound of Formula (I),

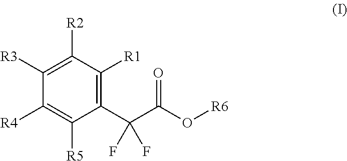

wherein:
each R1, R2, R4, and R5 is independently selected from the group consisting of hydrogen, halogen, amino, cyano, nitro, hydroxyl, C1-C5 alkyl, C3-6 cycloalkyl, C1-C4 alkoxy, C2-C3 alkenyl, C2-C3 alkynyl, C1-C2 haloalkoxy, halophenyl, C1-2 haloalkyl, C1-2alkoxyC1-2alkyl, C1-2 alkoxycarbonyl, C1-C2 alkylsulfanyl, C1-C2 alkylsulphinyl and C1-C2 alkylsulfonyl, wherein no more than one of R1, R2, R4, and R5 is C1-C4 alkoxy, or
R1 and R2 together with the carbon atoms to which they are attached form a 5- or 6-membered ring, wherein the 6-membered ring contains zero, one or two nitrogen atoms with the proviso that any nitrogen in the 6-membered ring is next to the benzene ring in structure (I), and wherein the 5-membered ring contains one or two heteroatoms independently selected from the group consisting of nitrogen and oxygen, and each R4, and R5 is independently selected from the group consisting of hydrogen, halogen and C1-C2 alkyl;
R3 is selected from the group consisting of hydrogen, halogen, amino, cyano, hydroxyl, C1-C5 alkyl, C1-C4 alkoxy, C2-C3 alkenyl, C2-C3 alkynyl, C1-C2 haloalkoxy and halophenyl;
at least one of R1, R2, R3, R4, and R5 is selected from the group consisting of amino, cyano, nitro, hydroxyl, C2-C5 alkyl, C3-6 cycloalkyl, C1-C4 alkoxy, C2-C3 alkenyl, C2-C3 alkynyl, C1-C2 haloalkoxy, halophenyl, C2 haloalkyl, C1-2alkoxyC1-2alkyl, C1-2 alkoxycarbonyl, C1-C2 alkylsulfanyl, C1-C2 alkylsulphinyl and C1-C2 alkylsulfonyl, or
R1 and R2 together with the carbon atoms to which they are attached form a 5- or 6-membered ring, wherein the 6-membered ring contains zero, one or two nitrogen atoms with the proviso that any nitrogen in the 6-membered ring is next to the benzene ring in structure (I), and wherein the 5-membered ring contains one or two heteroatoms independently selected from the group consisting of nitrogen and oxygen, and each R4, and R5 is independently selected from the group consisting of hydrogen, halogen and C1-C2 alkyl; and
R6 is selected from the group consisting of hydrogen, benzyl and C1-C3alkyl;
or an agronomically acceptable salt of said compound, as a herbicide.

According to a second aspect of the invention, there is provided an agrochemical composition comprising a herbicidally effective amount of a compound of formula (I) and an agrochemically-acceptable diluent or carrier. Such an agricultural composition may further comprise at least one additional active ingredient.

According to a third aspect of the invention, there is provided a method of controlling or preventing undesirable plant growth, wherein a herbicidally effective amount of a compound of formula (I), or a composition comprising this compound as active ingredient, is applied to the plants, to parts thereof or the locus thereof.

As used herein, the term “halogen” or “halo” refers to fluorine (fluoro), chlorine (chloro), bromine (bromo) or iodine (iodo), preferably fluorine, chlorine or bromine.

As used herein, cyano means a —CN group.

As used herein, hydroxy or hydroxyl means an —OH group.

As used herein, nitro means an —NO₂ group.

As used herein, amino means an —NH₂ group.

As used herein, the term “C₁-C₅ alkyl” refers to a straight or branched hydrocarbon chain radical consisting solely of carbon and hydrogen atoms, containing no unsaturation, having from one to five carbon atoms, and which is attached to the rest of the molecule by a single bond. C₁-C₃alkyl and C₁-C₂alkyl are to be construed accordingly. Examples of C₁-C₅alkyl include, but are not limited to, methyl (Me), ethyl (Et), n-propyl, 1-methylethyl (iso-propyl), n-butyl, and 1-dimethylethyl (t-butyl).

As used herein, the term “C₃₋₈ cycloalkyl” refers to a stable, monocyclic ring radical which is saturated and contains 3 to 8 carbon atoms. C₃₋₆cycloalkyl is to be construed accordingly. Examples of C₃₋₈cycloalkyl include, but are not limited to, cyclopropyl, cyclobutyl, cyclopentyl and cyclohexyl.

As used herein, the term “C₁-C₄ alkoxy” refers to a radical of the formula —ORₐ where Rₐ is a C₁-C₄alkyl radical as generally defined above. C₁-C₂ alkoxy is to be construed accordingly. Examples of C₁₋₄alkoxy include, but are not limited to, methoxy, ethoxy, propoxy, iso-propoxy and t-butoxy.

As used herein, the term “C₁₋₄ alkoxyC₁₋₄alkyl” refers to a radical of the formula Rb—O—Ra- wherein Rb is a C₁₋₄alkyl radical as generally defined above, and Ra is a C₁₋₄alkylene radical as generally defined above.

As used herein, the term “C₁-C₂ haloalkyl” refers to a C₁-C₂alkyl radical as generally defined above substituted by one or more of the same or different halogen atoms. C₂haloalkyl is to be construed accordingly. Examples of C₁-C₂haloalkyl include, but are not limited to chloromethyl, fluoromethyl, fluoroethyl, difluoromethyl, trifluoromethyl and 2,2,2-trifluoroethyl.

As used herein, the term “C₂-C₃ alkenyl” refers to a straight or branched hydrocarbon chain radical group consisting solely of carbon and hydrogen atoms, containing at least one double bond that can be of either the (E)- or (Z)-configuration, having from two to three carbon atoms, which is attached to the rest of the molecule by a single bond. Examples of C₂-C₃alkenyl include, but are not limited to ethenyl, prop-1-enyl and allyl (prop-2-enyl).

As used herein, the term “C₂-C₃ alkynyl” refers to a straight or branched hydrocarbon chain radical group consisting solely of carbon and hydrogen atoms, containing at least one triple bond, having from two to three carbon atoms, and which is attached to the rest of the molecule by a single bond. Examples of C₂-C₃alkynyl include, but are not limited to ethynyl, prop-1-ynyl and propargyl (prop-2-ynyl).

As used herein, the term “C₁-C₂ haloalkoxy” refers to a C₁-C₂alkoxy group as defined above substituted by one or more of the same or different halogen atoms. Examples of C₁-C₂haloalkoxy include, but are not limited to fluoromethoxy, difluoromethoxy, fluoroethoxy, trifluoromethoxy and trifluoroethoxy.

As used herein, the term “C₁₋₂ alkylsulfanyl” refers to a radical of the formula —SRₐ wherein Rₐ is a C₁₋₂alkyl radical as generally defined above.

As used herein, the term “C₁₋₂ alkylsulfinyl” refers to a radical of the formula —S(O)Rₐ wherein Rₐ is a C₁₋₂alkyl radical as generally defined above.

As used herein, the term “C₁₋₂ alkylsulfonyl” refers to a radical of the formula —S(O)₂Rₐ wherein Rₐ is a C₁₋₂alkyl radical as generally defined above.

As used herein, the term “C₁₋₄alkoxycarbonyl” refers to a radical of the formula RaOC(O)—, wherein Ra is a C₁₋₄alkyl radical as generally defined above.

As used herein, the term “C₁₋₃alkoxycarbonylC₁₋₃alkyl” refers to a radical of the formula —R_{b}C(O)ORₐ, wherein Rₐ is a C₁₋₃ alkyl radical as generally defined above and R_{b} is a C₁₋₃alkylene radical as generally defined above.

The presence of one or more possible asymmetric carbon atoms in a compound of formula (I) means that the compounds may occur in chiral isomeric forms, i.e., enantiomeric or diastereomeric forms. Also atropisomers may occur as a result of restricted rotation about a single bond. Formula (I) is intended to include all those possible isomeric forms and mixtures thereof. The present invention includes all those possible isomeric forms and mixtures thereof for a compound of formula (I). Likewise, formula (I) is intended to include all possible tautomers (including lactam-lactim tautomerism and keto-enol tautomerism) where present. The present invention includes all possible tautomeric forms for a compound of formula (I). Similarly, where there are di-substituted alkenes, these may be present in E or Z form or as mixtures of both in any proportion. The present invention includes all these possible isomeric forms and mixtures thereof for a compound of formula (I).

The compounds of formula (I) will typically be provided in the form of an agronomically acceptable salt, a zwitterion or an agronomically acceptable salt of a zwitterion. This invention covers all such agronomically acceptable salts, zwitterions and mixtures thereof in all proportions.

Suitable agronomically acceptable salts of the present invention can be with cations that include but are not limited to, metals, conjugate acids of amines and organic cations. Examples of suitable metals include aluminium, calcium, cesium, copper, lithium, magnesium, manganese, potassium, sodium, iron and zinc. Examples of suitable amines include allylamine, ammonia, amylamine, arginine, benethamine, benzathine, butenyl-2-amine, butylamine, butylethanolamine, cyclohexylamine, decylamine, diamylamine, dibutylamine, diethanolamine, diethylamine, diethylenetriamine, diheptylamine, dihexylamine, diisoamylamine, diisopropylamine, dimethylamine, dioctylamine, dipropanolamine, dipropargylamine, dipropylamine, dodecylamine, ethanolamine, ethylamine, ethylbutylamine, ethylenediamine, ethylheptylamine, ethyloctylamine, ethylpropanolamine, heptadecylamine, heptylamine, hexadecylamine, hexenyl-2-amine, hexylamine, hexylheptylamine, hexyloctylamine, histidine, indoline, isoamylamine, isobutanolamine, isobutylamine, isopropanolamine, isopropylamine, lysine, meglumine, methoxyethylamine, methylamine, methylbutylamine, methylethylamine, methylhexylamine, methylisopropylamine, methylnonylamine, methyloctadecylamine, methylpentadecylamine, morpholine, N,N-diethylethanolamine, N-methylpiperazine, nonylamine, octadecylamine, octylamine, oleylamine, pentadecylamine, pentenyl-2-amine, phenoxyethylamine, picoline, piperazine, piperidine, propanolamine, propylamine, propylenediamine, pyridine, pyrrolidine, sec-butylamine, stearylamine, tallowamine, tetradecylamine, tributylamine, tridecylamine, trimethylamine, triheptylamine, trihexylamine, triisobutylamine, triisodecylamine, triisopropylamine, trimethylamine, tripentylamine, tripropylamine, tris(hydroxymethyl)aminomethane, and undecylamine. Examples of suitable organic cations include benzyltributylammonium, benzyltrimethylammonium, benzyltriphenylphosphonium, choline, tetrabutylammonium, tetrabutylphosphonium, tetraethylammonium, tetraethylphosphonium, tetramethylammonium, tetramethylphosphonium, tetrapropylammonium, tetrapropylphosphonium, tributylsulfonium, tributylsulfoxonium, triethylsulfonium, triethylsulfoxonium, trimethylsulfonium, trimethylsulfoxonium, tripropylsulfonium and tripropylsulfoxonium.

In a preferred embodiment, the agrochemically acceptable salt is selected from the group consisting of sodium, potassium, aluminium, dimethylamine (DMA), diglycolamine (DGA) and choline salt. In an especially preferred embodiment, the agrochemically acceptable salt is choline salt.

The following list provides definitions, including preferred definitions, for substituents R¹, R², R³, R⁴, R⁵ and R⁶ with reference to the compounds of formula (I) according to the invention. For any one of these substituents, any of the definitions given below may be combined with any definition of any other substituent given below or elsewhere in this document.

Preferably R¹ is selected from the group consisting of hydrogen, halogen, amino, cyano, nitro, hydroxyl, C₁-C₅alkyl, C₃-C₄cycloalkyl, C₁-C₄alkoxy, C₂-C₃alkenyl, C₂-C₃alkynyl, C₁-C₂haloalkoxy, halophenyl and C₁₋₂alkylsulfanyl, more preferably hydrogen, fluorine, chlorine, nitro, cyano, C₁-C₄alkyl, cyclopropyl, methylsulfanyl, amino and C₁-C₃alkoxy, most preferably hydrogen, chlorine, cyano, nitro, methoxy, ethyl, cyclopropyl, ethoxy and isopropoxy.

Preferably R² is selected from the group consisting of hydrogen, halogen, amino, cyano, nitro, hydroxyl, C₁-C₅alkyl, C₁-C₄alkoxy, C₂-C₃alkenyl, C₂-C₃alkynyl, C₁-C₂haloalkoxy, halophenyl and C₁₋₂alkylsulfanyl, more preferably hydrogen, bromine, fluorine, chlorine, cyano, ethenyl, ethynyl, methyl, ethyl, methylsulfanyl, methoxy, amino and fluorophenyl, most preferably hydrogen, chlorine, cyano, ethynyl, methylsulfanyl and methoxy.

Preferably R1 and R2 together with the carbon atoms to which they are attached form a 5-membered ring containing one or two heteroatoms independently selected from the group consisting of nitrogen and oxygen, more preferably the 5-membered ring contains two oxygen atoms. Preferably the 5-membered ring is partially saturated, most preferably the 5-membered ring is saturated. Preferably the 5-membered ring is substituted by one or two substituents independently selected from the group consisting of fluorine, chlorine and methyl, more preferably the 5-membered ring is substituted by two fluorine.

Preferably R1 and R2 together with the carbon atoms to which they are attached form a 6-membered ring containing zero, one or two nitrogen atoms, with the proviso that any nitrogen in the 6-membered ring is next to the benzene ring in structure (I), more preferably the 6-membered ring contains zero or one nitrogen. Preferably the 6-membered ring is aromatic.

Preferably the 6-membered ring is substituted by zero or one substituent. If substituted, the substituent is preferably chlorine.

Preferably R³ is selected from the group consisting of hydrogen, halogen, amino, cyano, hydroxyl, C₁-C₅alkyl, C₁-C₄alkoxy, C₂-C₃alkenyl, C₂-C₃alkynyl, C₁-C₂haloalkoxy and halophenyl, more preferably hydrogen, amino, trifluoromethoxy, methyl, tert-butyl and methoxy, most preferably hydrogen.

Preferably R⁴ is selected from the group consisting of hydrogen, amino, fluorine, chlorine, methoxy, more preferably hydrogen.

Preferably R⁵ is selected from the group consisting of hydrogen, fluorine, chlorine, amino, nitro and methoxy, more preferably hydrogen, chlorine and methoxy.

Preferably R⁶ is selected from the group consisting of hydrogen and C₁-C₃alkyl, more preferably hydrogen.

A preferred subset of compounds is one in which;
each R1, R2, R4, and R5 is independently selected from the group consisting of hydrogen, halogen, amino, cyano, nitro, hydroxyl, C₁-C₅alkyl, C3-6cycloalkyl, C₁-C₄alkoxy, C₂-C₃alkenyl, C₂-C₃alkynyl, C₁-C₂haloalkoxy, halophenyl, C1-2alkoxyC1-2alkyl, C1-2 alkoxycarbonyl and C₁₋₂alkylsulfanyl, wherein no more than one of R1, R2, R4, and R5 is C₁-C₄alkoxy; or
R1 and R2 together with the carbon atoms to which they are attached form a 5- or 6-membered ring, wherein the 6-membered ring contains zero, one or two nitrogen atoms with the proviso that any nitrogen in the 6-membered ring is next to the benzene ring in structure (I), and wherein the 5-membered ring contains one or two heteroatoms selected from the group consisting of nitrogen and oxygen, and each R4, and R5 is independently selected from the group consisting of hydrogen, halogen and C₁-C₂alkyl;
R3 is selected from the group consisting of hydrogen, halogen, amino, cyano, hydroxyl, C₁-C₅alkyl, C₁-C₄alkoxy, C₂-C₃alkenyl, C₂-C₃alkynyl, C₁-C₂haloalkoxy and halophenyl;
at least one of R1, R2, R3, R4, and R5 is selected from the group consisting of amino, cyano, nitro, hydroxyl, C₂-C₅alkyl, cyclopropyl, C₁-C₄alkoxy, C₂-C₃alkenyl, C₂-C₃alkynyl, C₁-C₂haloalkoxy, halophenyl, C1-2alkoxyC1-2alkyl, C1-2 alkoxycarbonyl and C₁-C₂alkylsulfanyl, or
R1 and R2 together with the carbon atoms to which they are attached form a 5- or 6-membered ring, wherein the 6-membered ring contains zero, one or two nitrogen atoms with the proviso that any nitrogen in the 6-membered ring is next to the benzene ring in structure (I), and wherein the 5-membered ring contains one or two heteroatoms selected from the group consisting of nitrogen and oxygen, and each R4, and R5 is independently selected from the group consisting of hydrogen, halogen and C₁-C₂alkyl; and
R6 is selected from the group consisting of hydrogen and C₁-C₃alkyl.
   A more preferred subset of compounds is one in which;
each R1, R2, R4, and R5 is independently selected from the group consisting of hydrogen, chlorine, cyano, nitro, methoxy, ethyl, ethoxy, isopropoxy, ethynyl and methylsulfanyl; or
R1 and R2 together with the carbon atoms to which they are attached form a 5- or 6-membered ring, wherein the 6-membered ring is aromatic and contains zero or one nitrogen with the proviso that any nitrogen in the 6-membered ring is next to the benzene ring in structure (I), and wherein the 5-membered ring is partially or fully saturated and contains two oxygen in the ring, and each R4, and R5 is independently selected from the group consisting of hydrogen, halogen and C1-C2alkyl;
R3 is hydrogen;
at least one of R1, R2, R3, R4, and R5 is selected from the group consisting of cyano, nitro, methoxy, ethyl, ethoxy, isopropoxy, ethynyl and methylsulfanyl, or
R1 and R2 together with the carbon atoms to which they are attached form a 5- or 6-membered ring, wherein the 6-membered ring contains zero, one or two nitrogen atoms with the proviso that any nitrogen in the 6-membered ring is next to the benzene ring in structure (I), and wherein the 5-membered ring contains one or two heteroatoms selected from the group consisting of nitrogen and oxygen, and each R4, and R5 is independently selected from the group consisting of hydrogen, halogen and C₁-C₂alkyl; and
R6 is hydrogen.

### Table of Examples

Table 1 below discloses 477 specific compounds of formula (I), designated compound numbers 1-1 to 1-477 respectively, wherein R⁶ is hydrogen.

TABLE 1

| Compound | | | | | |
|---|---|---|---|---|---|
| Number | R¹ | R² | R³ | R⁴ | R⁵ |
| 1-1 | H | H | t-Bu | H | H |
| 1-2 | H | H | OMe | H | H |
| 1-3 | H | H | OCF3 | H | H |
| 1-4 | H | H | NH2 | H | H |
| 1-5 | H | H | H | OH | H |
| 1-6 | H | H | H | OMe | H |
| 1-7 | H | H | Me | OMe | H |
| 1-8 | H | H | H | NH2 | H |
| 1-9 | H | H | H | H | OMe |
| 1-10 | H | H | H | H | NH2 |
| 1-11 | H | F | OMe | H | H |
| 1-12 | H | F | NH2 | H | H |
| 1-13 | H | F | H | H | OMe |
| 1-14 | H | F | H | H | NH2 |
| 1-15 | H | Cl | OMe | H | H |
| 1-16 | H | Cl | NH2 | H | H |
| 1-17 | H | Cl | H | H | OMe |
| 1-18 | H | Cl | H | H | NH2 |
| 1-19 | H | Br | OMe | H | H |
| 1-20 | H | Br | OCF3 | H | H |
| 1-21 | H | Br | NH2 | H | H |
| 1-22 | H | Br | H | H | OMe |
| 1-23 | H | Br | H | H | NH2 |
| 1-24 | H | Me | OMe | H | H |
| 1-25 | H | Me | H | H | OMe |
| 1-26 | H | Me | H | H | NH2 |
| 1-27 | H | CF3 | OMe | H | H |
| 1-28 | H | CF3 | H | H | OMe |
| 1-29 | H | CF3 | H | H | NH2 |
| 1-30 | F | H | OMe | H | H |
| 1-31 | F | H | OMe | Cl | H |
| 1-32 | F | H | OCHF2 | H | H |
| 1-33 | F | H | OCF3 | H | H |
| 1-34 | F | H | NH2 | H | H |
| 1-35 | F | H | H | OMe | H |
| 1-36 | F | H | H | NH2 | H |
| 1-37 | F | H | H | H | OMe |
| 1-38 | F | H | H | H | NH2 |
| 1-39 | F | F | OMe | H | H |
| 1-40 | F | F | OCF3 | H | H |
| 1-41 | F | F | NH2 | H | H |
| 1-42 | F | F | H | H | OMe |
| 1-43 | F | F | H | H | NH2 |
| 1-44 | F | Cl | OMe | H | H |
| 1-45 | F | Cl | OCF3 | H | H |
| 1-46 | F | Cl | NH2 | H | H |
| 1-47 | F | Cl | H | H | OMe |
| 1-48 | F | Cl | H | H | NH2 |
| 1-49 | F | Br | OMe | H | H |
| 1-50 | F | Br | H | H | OMe |
| 1-51 | F | Br | H | H | NH2 |
| 1-52 | F | Me | OMe | H | H |
| 1-53 | F | Me | H | H | OMe |
| 1-54 | F | Me | H | H | NH2 |
| 1-55 | F | CF3 | OMe | H | H |
| 1-56 | F | CF3 | H | H | OMe |
| 1-57 | F | CF3 | H | H | NH2 |
| 1-58 | Cl | H | OMe | H | H |
| 1-59 | Cl | H | OCF3 | H | H |
| 1-60 | Cl | H | NH2 | H | H |
| 1-61 | Cl | H | H | OH | H |
| 1-62 | Cl | H | H | OMe | H |
| 1-63 | Cl | H | H | NH2 | H |
| 1-64 | Cl | H | H | 4-fluorophenyl | H |
| 1-65 | Cl | H | H | H | OMe |
| 1-66 | Cl | H | H | H | NH2 |
| 1-67 | Cl | H | H | H | NO2 |
| 1-68 | Cl | F | OMe | H | H |
| 1-69 | Cl | F | OCF3 | H | H |
| 1-70 | Cl | F | NH2 | H | H |
| 1-71 | Cl | F | H | H | OMe |
| 1-72 | Cl | F | H | H | NH2 |
| 1-73 | Cl | Cl | OMe | H | H |
| 1-74 | Cl | Cl | OCF3 | H | H |
| 1-75 | Cl | Cl | NH2 | H | H |
| 1-76 | Cl | Cl | H | OMe | H |
| 1-77 | Cl | Cl | H | NH3Cl | H |
| 1-78 | Cl | Cl | H | 4-fluorophenyl | H |
| 1-79 | Cl | Cl | H | H | OMe |
| 1-80 | Cl | Cl | H | H | NH2 |
| 1-81 | Cl | Cl | H | Cl | NH2 |
| 1-82 | Cl | Cl | H | Cl | OMe |
| 1-83 | Cl | Br | OMe | H | H |
| 1-84 | Cl | Br | OCF3 | H | H |
| 1-85 | Cl | Br | NH2 | H | H |
| 1-86 | Cl | Br | H | OMe | H |
| 1-87 | Cl | Br | H | NH2 | H |
| 1-88 | Cl | Br | H | H | OMe |
| 1-89 | Cl | Br | H | H | NH2 |
| 1-90 | Cl | Me | OMe | H | H |
| 1-91 | Cl | Me | OCF3 | H | H |
| 1-92 | Cl | Me | NH2 | H | H |
| 1-93 | Cl | Me | H | H | OMe |
| 1-94 | Cl | Me | H | H | NH2 |
| 1-95 | Cl | CF3 | OMe | H | H |
| 1-96 | Cl | CF3 | H | H | OMe |
| 1-97 | Cl | CF3 | H | H | NH2 |
| 1-98 | Br | H | OMe | H | H |
| 1-99 | Br | H | OCF3 | H | H |
| 1-100 | Br | H | NH2 | H | H |
| 1-101 | Br | H | H | OMe | H |
| 1-102 | Br | H | H | NH2 | H |
| 1-103 | Br | H | H | H | OMe |
| 1-104 | Br | H | H | H | NH2 |
| 1-105 | Br | F | OMe | H | H |
| 1-106 | Br | F | H | H | OMe |
| 1-107 | Br | F | H | H | NH2 |
| 1-108 | Br | Cl | OMe | H | H |
| 1-109 | Br | Cl | OCF3 | H | H |
| 1-110 | Br | Cl | NH2 | H | H |
| 1-111 | Br | Cl | H | OMe | H |
| 1-112 | Br | Cl | H | NH2 | H |
| 1-113 | Br | Cl | H | H | OMe |
| 1-114 | Br | Cl | H | H | NH2 |
| 1-115 | Br | Me | OMe | H | H |
| 1-116 | Br | Me | OCF3 | H | H |
| 1-117 | Br | Me | NH2 | H | H |
| 1-118 | Br | Me | H | H | OMe |
| 1-119 | Br | Me | H | H | NH2 |
| 1-120 | Me | H | OMe | H | H |
| 1-121 | Me | H | OCF3 | H | H |
| 1-122 | Me | H | NH2 | H | H |
| 1-123 | Me | H | H | OMe | H |
| 1-124 | Me | H | H | NH2 | H |
| 1-125 | Me | H | H | H | OMe |
| 1-126 | Me | H | H | H | NH2 |
| 1-127 | Me | F | OMe | H | H |
| 1-128 | Me | F | OCF3 | H | H |
| 1-129 | Me | F | NH2 | H | H |
| 1-130 | Me | F | H | OMe | H |
| 1-131 | Me | F | H | NH2 | H |
| 1-132 | Me | F | H | H | OMe |
| 1-133 | Me | F | H | H | NH2 |
| 1-134 | Me | Cl | OMe | H | H |
| 1-135 | Me | Cl | OCF3 | H | H |
| 1-136 | Me | Cl | NH2 | H | H |
| 1-137 | Me | Cl | H | OMe | H |
| 1-138 | Me | Cl | H | NH2 | H |
| 1-139 | Me | Cl | H | H | OMe |
| 1-140 | Me | Cl | H | H | NH2 |
| 1-141 | Me | Br | OMe | H | H |
| 1-142 | Me | Br | OCF3 | H | H |
| 1-143 | Me | Br | NH2 | H | H |
| 1-144 | Me | Br | H | OMe | H |
| 1-145 | Me | Br | H | NH2 | H |
| 1-146 | Me | Br | H | H | OMe |
| 1-147 | Me | Br | H | H | NH2 |
| 1-148 | Me | Me | OMe | H | H |
| 1-149 | Me | Me | OCF3 | H | H |
| 1-150 | Me | Me | NH2 | H | H |
| 1-151 | Me | Me | H | OMe | H |
| 1-152 | Me | Me | H | NH2 | H |
| 1-153 | Me | Me | H | H | OMe |
| 1-154 | Me | Me | H | H | NH2 |
| 1-155 | Me | CF3 | OMe | H | H |
| 1-156 | Me | CF3 | OCF3 | H | H |
| 1-157 | Me | CF3 | NH2 | H | H |
| 1-158 | Me | CF3 | H | OMe | H |
| 1-159 | Me | CF3 | H | NH2 | H |
| 1-160 | Me | CF3 | H | H | OMe |
| 1-161 | Me | CF3 | H | H | NH2 |
| 1-162 | CF3 | H | OMe | H | H |
| 1-163 | CF3 | H | OCF3 | H | H |
| 1-164 | CF3 | H | NH2 | H | H |
| 1-165 | CF3 | H | H | OMe | H |
| 1-166 | CF3 | H | H | NH2 | H |
| 1-167 | CF3 | H | H | H | OMe |
| 1-168 | CF3 | H | H | H | NH2 |
| 1-169 | CF3 | F | OMe | H | H |
| 1-170 | CF3 | F | OCF3 | H | H |
| 1-171 | CF3 | F | NH2 | H | H |
| 1-172 | CF3 | F | H | OMe | H |
| 1-173 | CF3 | F | H | NH2 | H |
| 1-174 | CF3 | F | H | H | OMe |
| 1-175 | CF3 | F | H | H | NH2 |
| 1-176 | CF3 | Cl | OMe | H | H |
| 1-177 | CF3 | Cl | OCF3 | H | H |
| 1-178 | CF3 | Cl | NH2 | H | H |
| 1-179 | CF3 | Cl | H | OMe | H |
| 1-180 | CF3 | Cl | H | NH2 | H |
| 1-181 | CF3 | Cl | H | H | OMe |
| 1-182 | CF3 | Cl | H | H | NH2 |
| 1-183 | CF3 | Me | OMe | H | H |
| 1-184 | CF3 | Me | H | H | OMe |
| 1-185 | CF3 | Me | H | H | NH2 |
| 1-186 | H | CN | H | H | H |
| 1-187 | F | Et | H | H | H |
| 1-188 | F | CH═CH2 | H | H | H |
| 1-189 | F | CCH | H | H | H |
| 1-190 | F | OMe | H | H | H |
| 1-191 | F | SMe | H | H | H |
| 1-192 | F | CN | H | H | H |
| 1-193 | F | SOMe | H | H | H |
| 1-194 | F | SO2Me | H | H | H |
| 1-195 | F | NH2 | H | H | H |
| 1-196 | Cl | Et | H | H | H |
| 1-197 | Cl | Et | F | H | H |
| 1-198 | Cl | Et | Cl | H | H |
| 1-199 | Cl | Et | Br | H | H |
| 1-200 | Cl | Et | Me | H | H |
| 1-201 | Cl | Et | CF3 | H | H |
| 1-202 | Cl | Et | H | F | H |
| 1-203 | Cl | Et | H | Cl | H |
| 1-204 | Cl | Et | H | H | F |
| 1-205 | Cl | Et | H | H | Cl |
| 1-206 | Cl | Et | H | H | Me |
| 1-207 | Cl | CH═CH2 | H | H | H |
| 1-208 | Cl | CCH | H | H | H |
| 1-209 | Cl | OMe | H | H | H |
| 1-210 | Cl | OMe | F | H | H |
| 1-211 | Cl | OMe | Cl | H | H |
| 1-212 | Cl | OMe | Br | H | H |
| 1-213 | Cl | OMe | Me | H | H |
| 1-214 | Cl | OMe | CF3 | H | H |
| 1-215 | Cl | OMe | H | F | H |
| 1-216 | Cl | OMe | H | Cl | H |
| 1-217 | Cl | OMe | H | H | F |
| 1-218 | Cl | OMe | H | H | Cl |
| 1-219 | Cl | OMe | H | H | Me |
| 1-220 | Cl | SMe | H | H | H |
| 1-221 | Cl | SMe | F | H | H |
| 1-222 | Cl | SMe | Cl | H | H |
| 1-223 | Cl | SMe | Br | H | H |
| 1-224 | Cl | SMe | Me | H | H |
| 1-225 | Cl | SMe | CF3 | H | H |
| 1-226 | Cl | SMe | H | F | H |
| 1-227 | Cl | SMe | H | Cl | H |
| 1-228 | Cl | SMe | H | H | F |
| 1-229 | Cl | SMe | H | H | Cl |
| 1-230 | Cl | SMe | H | H | Me |
| 1-231 | Cl | CN | H | H | H |
| 1-232 | Cl | SOMe | H | H | H |
| 1-233 | Cl | SO2Me | H | H | H |
| 1-234 | Cl | NH2 | H | H | H |
| 1-235 | Cl | NH2 | H | Cl | H |
| 1-236 | Cl | NH2 | H | H | Cl |
| 1-237 | Cl | NO2 | H | H | H |
| 1-238 | Cl | NO2 | H | Cl | H |
| 1-239 | Me | Et | H | H | H |
| 1-240 | Me | CH═CH2 | H | H | H |
| 1-241 | Me | CCH | H | H | H |
| 1-242 | Me | OMe | H | H | H |
| 1-243 | Me | SMe | H | H | H |
| 1-244 | Me | CN | H | H | H |
| 1-245 | Me | SOMe | H | H | H |
| 1-246 | Me | SO2Me | H | H | H |
| 1-247 | Me | NH2 | H | H | H |
| 1-248 | NO2 | H | H | H | H |
| 1-249 | NO2 | F | H | H | H |
| 1-250 | NO2 | Cl | H | H | H |
| 1-251 | NH2 | H | H | Cl | H |
| 1-252 | NH2 | Cl | H | H | H |
| 1-253 | NH2 | Cl | H | Cl | H |
| 1-254 | NH2 | Cl | H | H | Cl |
| 1-255 | CN | H | H | H | H |
| 1-256 | CN | H | F | H | H |
| 1-257 | CN | H | Cl | H | H |
| 1-258 | CN | H | H | H | F |
| 1-259 | CN | H | H | H | Cl |
| 1-260 | CN | H | H | H | Me |
| 1-261 | CN | F | H | H | H |
| 1-262 | CN | Cl | H | H | H |
| 1-263 | Et | H | H | H | H |
| 1-264 | Et | H | F | H | H |
| 1-265 | Et | H | Cl | H | H |
| 1-266 | Et | H | Br | H | H |
| 1-267 | Et | H | Me | H | H |
| 1-268 | Et | H | CF3 | H | H |
| 1-269 | Et | H | H | F | H |
| 1-270 | Et | H | H | Cl | H |
| 1-271 | Et | H | H | H | F |
| 1-272 | Et | H | H | H | Cl |
| 1-273 | Et | H | H | H | Me |
| 1-274 | Et | F | H | H | H |
| 1-275 | Et | Cl | H | H | H |
| 1-276 | Et | Cl | F | H | H |
| 1-277 | Et | Cl | Cl | H | H |
| 1-278 | Et | Cl | Br | H | H |
| 1-279 | Et | Cl | Me | H | H |
| 1-280 | Et | Cl | CF3 | H | H |
| 1-281 | Et | Cl | H | F | H |
| 1-282 | Et | Cl | H | Cl | H |
| 1-283 | Et | Cl | H | H | F |
| 1-284 | Et | Cl | H | H | Cl |
| 1-285 | Et | Cl | H | H | Me |
| 1-286 | CH═CH2 | H | H | H | H |
| 1-287 | CH═CH2 | F | H | H | H |
| 1-288 | CH═CH2 | Cl | H | H | H |
| 1-289 | CCH | H | H | H | H |
| 1-290 | CCH | F | H | H | H |
| 1-291 | CCH | Cl | H | H | H |
| 1-292 | CCH | Cl | F | H | H |
| 1-293 | CCH | Cl | Cl | H | H |
| 1-294 | CCH | Cl | H | H | F |
| 1-295 | CCH | Cl | H | H | Cl |
| 1-296 | CCH | Cl | H | H | Me |
| 1-297 | OH | F | H | H | F |
| 1-298 | OH | H | Cl | Cl | H |
| 1-299 | OMe | H | F | H | H |
| 1-300 | OMe | H | Cl | H | H |
| 1-301 | OMe | H | Br | H | H |
| 1-302 | OMe | H | Me | H | H |
| 1-303 | OMe | H | CF3 | H | H |
| 1-304 | OMe | H | H | F | H |
| 1-305 | OMe | H | H | Cl | H |
| 1-306 | OMe | F | H | H | H |
| 1-307 | OMe | F | F | H | H |
| 1-308 | OMe | F | Cl | H | H |
| 1-309 | OMe | F | Br | H | H |
| 1-310 | OMe | F | Me | H | H |
| 1-311 | OMe | F | CF3 | H | H |
| 1-312 | OMe | F | H | F | H |
| 1-313 | OMe | F | H | Cl | H |
| 1-314 | OMe | F | H | H | F |
| 1-315 | OMe | F | H | H | Cl |
| 1-316 | OMe | F | H | H | Me |
| 1-317 | OMe | Cl | H | H | H |
| 1-318 | OMe | Cl | F | H | H |
| 1-319 | OMe | Cl | Cl | H | H |
| 1-320 | OMe | Cl | Br | H | H |
| 1-321 | OMe | Cl | Me | H | H |
| 1-322 | OMe | Cl | CF3 | H | H |
| 1-323 | OMe | Cl | H | F | H |
| 1-324 | OMe | Cl | H | Cl | H |
| 1-325 | OMe | Cl | H | H | F |
| 1-326 | OMe | Cl | H | H | Cl |
| 1-327 | OMe | Cl | H | H | Me |
| 1-328 | OEt | H | H | H | H |
| 1-329 | OEt | H | F | H | H |
| 1-330 | OEt | H | Cl | H | H |
| 1-331 | OEt | H | Br | H | H |
| 1-332 | OEt | H | Me | H | H |
| 1-333 | OEt | H | CF3 | H | H |
| 1-334 | OEt | H | H | F | H |
| 1-335 | OEt | H | H | Cl | H |
| 1-336 | OEt | H | H | H | F |
| 1-337 | OEt | H | H | H | Cl |
| 1-338 | OEt | H | H | H | Me |
| 1-339 | OEt | F | H | H | H |
| 1-340 | OEt | Cl | H | H | H |
| 1-341 | O—iPr | H | H | H | H |
| 1-342 | O—iPr | F | H | H | H |
| 1-343 | O—iPr | Cl | H | H | H |
| 1-344 | SMe | H | H | H | H |
| 1-345 | SMe | H | F | H | H |
| 1-346 | SMe | H | Cl | H | H |
| 1-347 | SMe | H | Br | H | H |
| 1-348 | SMe | H | Me | H | H |
| 1-349 | SMe | H | CF3 | H | H |
| 1-350 | SMe | H | H | F | H |
| 1-351 | SMe | H | H | Cl | H |
| 1-352 | SMe | H | H | H | F |
| 1-353 | SMe | H | H | H | Cl |
| 1-354 | SMe | H | H | H | Me |
| 1-355 | SMe | F | H | H | H |
| 1-356 | SMe | Cl | H | H | H |
| 1-357 | SMe | Cl | F | H | H |
| 1-358 | SMe | Cl | Cl | H | H |
| 1-359 | SMe | Cl | Br | H | H |
| 1-360 | SMe | Cl | Me | H | H |
| 1-361 | SMe | Cl | CF3 | H | H |
| 1-362 | SMe | Cl | H | F | H |
| 1-363 | SMe | Cl | H | Cl | H |
| 1-364 | SMe | Cl | H | H | F |
| 1-365 | SMe | Cl | H | H | Cl |
| 1-366 | SMe | Cl | H | H | Me |
| 1-367 | SOMe | H | H | H | H |
| 1-368 | SOMe | F | H | H | H |
| 1-369 | SOMe | Cl | H | H | H |
| 1-370 | SOMe | Br | H | H | H |
| 1-371 | SOMe | Me | H | H | H |
| 1-372 | SOMe | CF3 | H | H | H |
| 1-373 | SO2Me | H | H | H | H |
| 1-374 | SO2Me | F | H | H | H |
| 1-375 | SO2Me | Cl | H | H | H |
| 1-376 | SO2Me | Br | H | H | H |
| 1-377 | SO2Me | Me | H | H | H |
| 1-378 | SO2Me | CF3 | H | H | H |
| 1-379 | —OCH2O— | | H | H | H |
| 1-380 | —OCH2O— | | F | H | H |
| 1-381 | —OCH2O— | | Cl | H | H |
| 1-382 | —OCH2O— | | H | H | F |
| 1-383 | —OCH2O— | | H | H | Cl |
| 1-384 | —OCH2O— | | H | H | Me |
| 1-385 | —OCF2O— | | H | H | H |
| 1-386 | —OCF2O— | | F | H | H |
| 1-387 | —OCF2O— | | Cl | H | H |
| 1-388 | —OCF2O— | | H | H | F |
| 1-389 | —OCF2O— | | H | H | Cl |
| 1-390 | —OCF2O— | | H | H | Me |
| 1-391 | —C(Me)═N—O— | | H | H | H |
| 1-392 | —C(Me)═N—O— | | F | H | H |
| 1-393 | —C(Me)═N—O— | | Cl | H | H |
| 1-394 | —C(Me)═N—O— | | H | H | F |
| 1-395 | —C(Me)═N—O— | | H | H | Cl |
| 1-396 | —C(Me)═N—O— | | H | H | Me |
| 1-397 | —CH═CH—CH═CH— | | H | H | H |
| 1-398 | —CH═CH—CH═CH— | | F | H | H |
| 1-399 | —CH═CH—CH═CH— | | Cl | H | H |
| 1-400 | —CH═CH—CH═CH— | | H | H | F |
| 1-401 | —CH═CH—CH═CH— | | H | H | Cl |
| 1-402 | —CH═CH—CH═CH— | | H | H | Me |
| 1-403 | —N═CH—CH═CH— | | H | H | H |
| 1-404 | —N═CH—CH═CH— | | F | H | H |
| 1-405 | —N═CH—CH═CH— | | Cl | H | H |
| 1-406 | —N═CH—CH═CH— | | H | H | F |
| 1-407 | —N═CH—CH═CH— | | H | H | Cl |
| 1-408 | —N═CH—CH═CH— | | H | H | Me |
| 1-409 | —N═CH—CH═N— | | H | H | H |
| 1-410 | —N═CH—CH═N— | | F | H | H |
| 1-411 | —N═CH—CH═N— | | Cl | H | H |
| 1-412 | —N═CH—CH═N— | | H | H | F |
| 1-413 | —N═CH—CH═N— | | H | H | Cl |
| 1-414 | —N═CH—CH═N— | | H | H | Me |
| 1-415 | —OCH2CH2O— | | H | H | H |
| 1-416 | —C(Cl)═CH—CH═CH— | | H | H | H |
| 1-417 | —N═CH—C(Cl)═CH— | | H | H | H |
| 1-418 | —N═CH—C(Cl)═CH— | | H | H | Cl |
| 1-419 | —C(CF2CO2Et)═CH—CH═CH— | | H | H | H |
| 1-420 | —CH2CH2O— | | H | H | H |
| 1-421 | —CH2CH2O— | | F | H | H |
| 1-422 | —CH2CH2O— | | Cl | H | H |
| 1-423 | —CH2CH2O— | | H | H | F |
| 1-424 | —CH2CH2O— | | H | H | Cl |
| 1-425 | —CH2CH2O— | | H | H | Me |
| 1-426 | cyclopropyl | H | H | H | H |
| 1-427 | cyclopropyl | F | H | H | H |
| 1-428 | cyclopropyl | Cl | H | H | H |
| 1-429 | cyclopropyl | Cl | F | H | H |
| 1-430 | cyclopropyl | Cl | Cl | H | H |
| 1-431 | cyclopropyl | Cl | H | H | F |
| 1-432 | cyclopropyl | Cl | H | H | Cl |
| 1-433 | cyclopropyl | Cl | H | H | Me |
| 1-434 | CH2OMe | H | H | H | H |
| 1-435 | CH2OMe | F | H | H | H |
| 1-436 | CH2OMe | Cl | H | H | H |
| 1-437 | CH2OMe | Cl | F | H | H |
| 1-438 | CH2OMe | Cl | Cl | H | H |
| 1-439 | CH2OMe | Cl | H | H | F |
| 1-440 | CH2OMe | Cl | H | H | Cl |
| 1-441 | CH2OMe | Cl | H | H | Me |
| 1-442 | CO2Me | H | H | H | H |
| 1-443 | CO2Me | F | H | H | H |
| 1-444 | CO2Me | Cl | H | H | H |
| 1-445 | CO2Me | Cl | F | H | H |
| 1-446 | CO2Me | Cl | Cl | H | H |
| 1-447 | CO2Me | Cl | H | H | F |
| 1-448 | CO2Me | Cl | H | H | Cl |
| 1-449 | CO2Me | Cl | H | H | Me |
| 1-450 | OCHF2 | H | H | H | H |
| 1-451 | OCHF2 | F | H | H | H |
| 1-452 | OCHF2 | Cl | H | H | H |
| 1-453 | OCHF2 | Cl | F | H | H |
| 1-454 | OCHF2 | Cl | Cl | H | H |
| 1-455 | OCHF2 | Cl | H | H | F |
| 1-456 | OCHF2 | Cl | H | H | Cl |
| 1-457 | OCHF2 | Cl | H | H | Me |
| 1-458 | F | OMe | F | H | H |
| 1-459 | Cl | OCHF2 | H | H | H |
| 1-460 | Cl | CN | F | H | H |
| 1-461 | NO2 | F | OMe | H | H |
| 1-462 | CN | Me | H | H | H |
| 1-463 | OMe | Me | H | H | H |
| 1-464 | OMe | CF3 | H | H | H |
| 1-465 | OCF3 | H | H | H | H |
| 1-466 | OCF3 | F | H | H | H |
| 1-467 | OCF3 | Cl | H | H | H |
| 1-468 | OCF3 | Cl | F | H | H |
| 1-469 | OCF3 | Cl | Cl | H | H |
| 1-470 | OCF3 | Cl | H | H | F |
| 1-471 | OCF3 | Cl | H | H | Cl |
| 1-472 | OCF3 | Cl | H | H | Me |
| 1-473 | H | CO2Me | H | H | H |
| 1-474 | H | H | CN | H | H |

| | | | | |
|---|---|---|---|---|
| 1-475 | —N═CH—C(F)═CH— | H | H | H |
| 1-476 | —N═CH—C(F)═CH— | H | H | Cl |
| 1-477 | —NH—CH═CH— | H | H | H |

477 compounds of formula (I), wherein R⁶ is methyl and the values of R¹-R⁵ are as given in Table 1 for compounds 1-1 to 1-477, are designated as compound numbers 2-1 to 2-477 respectively.

477 compounds of formula (I), wherein R⁶ is ethyl and the values of R¹-R⁵ are as given in Table 1 for compounds 1-1 to 1-477, are designated as compound numbers 3-1 to 3-477 respectively.

477 compounds of formula (I), wherein R⁶ is benzyl and the values of R¹-R⁵ are as given in Table 1 for compounds 1-1 to 1-477, are designated as compound numbers 4-1 to 4-477 respectively.

477 compounds of formula (I), wherein R⁶ is lithium and the values of R¹-R⁵ are as given in Table 1 for compounds 1-1 to 1-477, are designated as compound numbers 5-1 to 5-477 respectively.

477 compounds of formula (I), wherein R⁶ is sodium and the values of R¹-R⁵ are as given in Table 1 for compounds 1-1 to 1-477, are designated as compound numbers 6-1 to 6-477 respectively.

477 compounds of formula (I), wherein R⁶ is ammonium and the values of R¹-R⁵ are as given in Table 1 for compounds 1-1 to 1-477, are designated as compound numbers 7-1 to 7-477 respectively.

477 compounds of formula (I), wherein R⁶ is diisopropylammonium and the values of R¹-R⁵ are as given in Table 1 for compounds 1-1 to 1-477, are designated as compound numbers 8-1 to 8-477 respectively.

477 compounds of formula (I), wherein R⁶ is N,N,N-trimethylethanolammonium and the values of R¹-R⁵ are as given in Table 1 for compounds 1-1 to 1-477, are designated as compound numbers 9-1 to 9-477 respectively.

Compounds of the invention may be prepared by techniques known to the person skilled in the art of organic chemistry. General methods for the production of compounds of formula (I) are described below. Unless otherwise stated in the text, the substituents R¹, R², R³, R⁴, R⁵ and R⁶ are as defined hereinbefore. The starting materials used for the preparation of the compounds of the invention may be purchased from usual commercial suppliers or may be prepared by known methods. The starting materials as well as the intermediates may be purified before use in the next step by state of the art methodologies such as chromatography, crystallization, distillation and filtration.

Compounds of formula (I) may be prepared from esters of formula (A) as shown in reaction scheme 1.

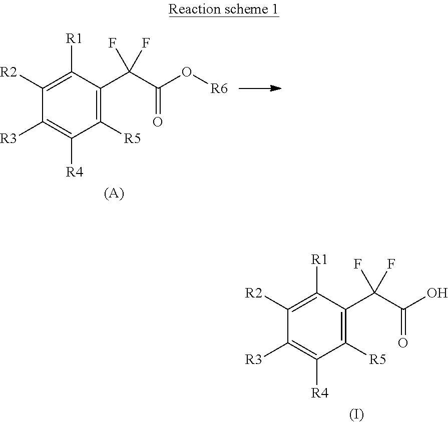

For example, a compound of formula (A), wherein R6 represents methyl or ethyl, may be treated with a base, such as lithium hydroxide, in a suitable solvent such as a mixture of ethanol and water.

Compounds of formula (A) may be prepared from aryl halides of formula (B) and alkyl halides of formula (C) as shown in reaction scheme 2.

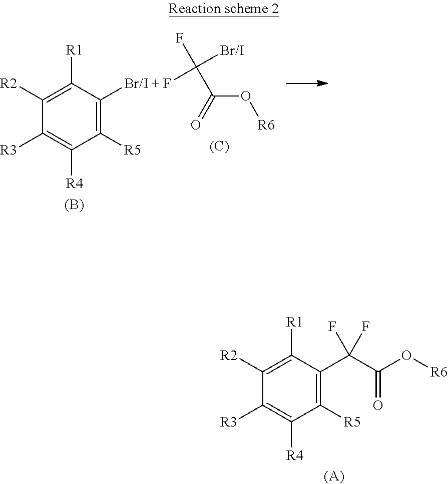

For example, a mixture of a compound of formula (B) and a compound of formula (C), wherein R6 represents methyl or ethyl, may be treated with a metal, such as copper, in a suitable solvent such as dimethyl sulfoxide.

Aryl bromides or aryl iodides of formula (B) are commercially available or may be prepared by methods well known in the literature.

Alkyl bromides or alkyl iodides of formula (C) are available or can be prepared by methods known in the literature.

Alternatively, compounds of formula (A) may be prepared from ketones of formula (D) as shown in reaction scheme 3.

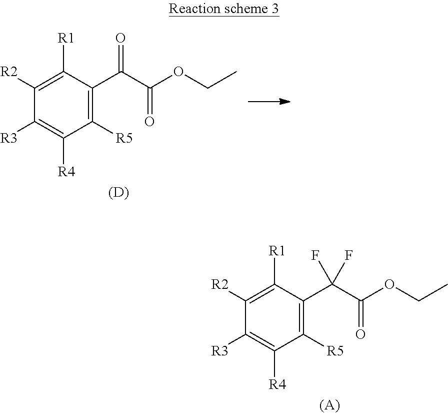

For example, a compound of formula (D) can be treated with a fluorinating agent, such as diethylaminosulfur trifluoride, in a suitable solvent, such as dichloromethane.

Ketones of formula (D) may be prepared from aryl halides of formula (E) as shown in reaction scheme 4.

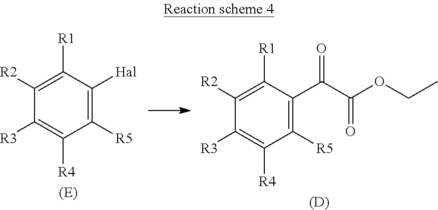

For example, a mixture of a compound of formula (E), wherein Hal represents a halogen atom, for example a chlorine, bromine or iodine atom, may be treated with a base, such as n-butyllithium, and a reagent, such as dimethyl oxalate or oxalyl chloride followed by ethanol, in a suitable solvent such as tetrahydrofuran.

Aryl halides of formula (E) are commercially available or may be prepared by methods well known in the literature.

Compounds of formula (A) may be prepared from esters of formula (F) as shown in reaction scheme 5.

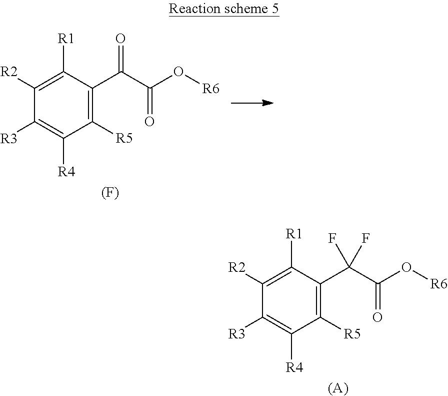

For example, a mixture of a compound of formula (F), wherein R6 represents methyl or ethyl, may be treated with a base, such as potassium bis(trimethylsilyl)amide, and a fluorinating reagent, such as N-fluorobenzenesulfonimide, in a suitable solvent such as tetrahydrofuran.

Compounds of formula (F) may be prepared from acids of formula (G) as shown in reaction scheme 6.

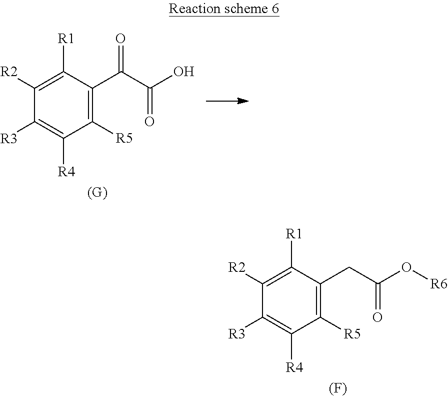

For example, a mixture of a compound of formula (G) may be treated with a chlorinating reagent, such as oxalyl chloride, and an alcohol, such as methanol, in a suitable solvent such as dichloromethane.

Compounds of formula (G) may be prepared from acids of formula (H) as shown in reaction scheme 7.

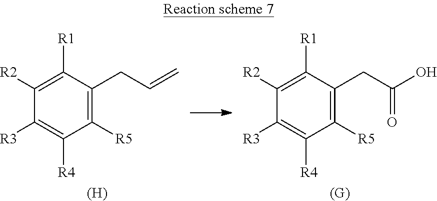

For example, a mixture of a compound of formula (H) may be treated with an oxidant, such as a mixture of ruthenium(III) chloride and sodium periodate, in a suitable solvent such as a mixture of water, acetonitrile and ethyl acetate, followed by addition of sodium metabisulfite in a suitable solvent such as water.

Allylic aromatic compounds of formula (H) are commercially available or may be prepared by methods well known in the literature.

Compounds of formula (I) may be prepared from esters of formula (A) as shown in reaction scheme 8.

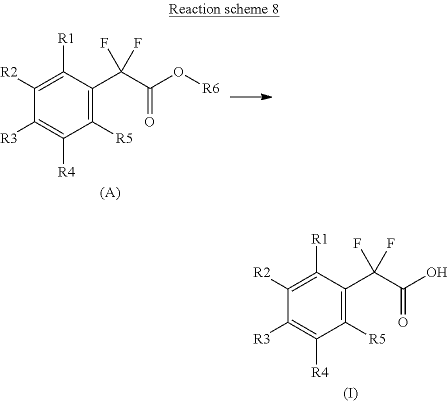

For example, a compound of formula (A), wherein R6 represents methyl or ethyl, may be treated with an acid, such as conc HCl, in a suitable solvent such as water.

Compounds of formula (A) may be prepared from aryl halides of formula (B) and alkyl halides of formula (C) as shown in reaction scheme 2.

Compounds of formula (A) may be prepared from aromatics of formula (J) and alkyl halides of formula (C) as shown in reaction scheme 9.

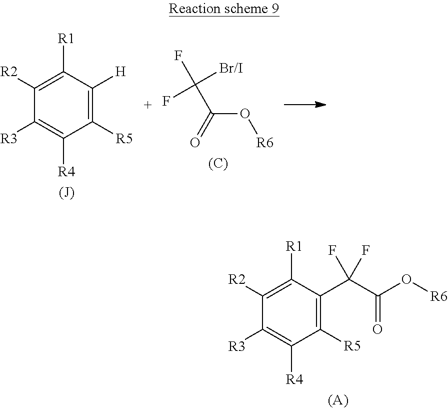

For example, a mixture of a compound of formula (C) and a compound of formula (J) may be treated with a metal complex, such as potassium phosphate, in a suitable solvent such as dimethyl sulfoxide with an Iridium complex.

Phenyl compounds of formula (J) and alkyl halides of formula (C) are commercially available or may be prepared by methods well known in the literature.

Compounds of formula (A) may be prepared from aromatics of formula (J) and fluoro-alkylsilanes of formula (K) as shown in reaction scheme 10.

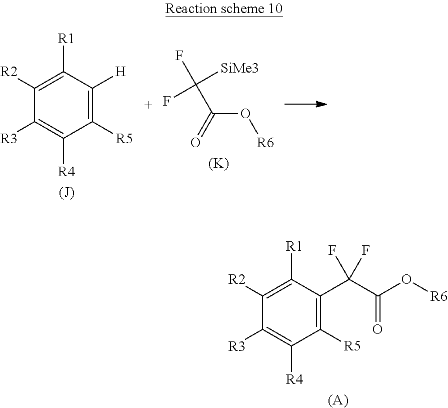

For example, a mixture of a compound of formula (J) and a compound of formula (K), wherein R6 represents methyl or ethyl, may be treated with a metal complex, such as potassium fluoride, in a suitable solvent such as dichloroethane with a Lewis acid such as silver trifluoromethanesulfonate.

Aryl species of formula (J) are commercially available or may be prepared by methods well known in the literature.

Fluoro-alkylsilanes of formula (K) are available or can be prepared by methods known in the literature.

Compounds of formula (A) may be prepared from aromatics of formula (J) and alkyl halides of formula (C) as shown in reaction scheme 11.

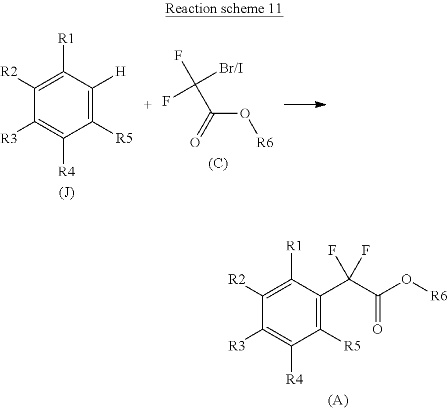

For example, a mixture of a compound of formula (J) and a compound of formula (C), wherein R6 represents methyl or ethyl, may be treated with a metal complex, such as ferrocene, and hydrogen peroxide in a suitable solvent such as dimethyl sulfoxide.

Aryl species of formula (J) are commercially available or may be prepared by methods well known in the literature.

Alkyl bromides or alkyl iodides of formula (C) are available or can be prepared by methods known in the literature. Compounds of formula (L) may be prepared from aryl bromides of formula (M) as shown in reaction scheme 12.

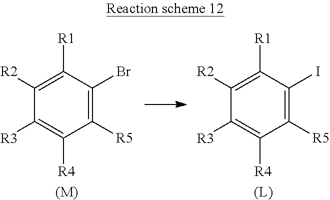

For example, a compound of formula (M) may be treated with a metal, such as copper (1) iodide, and an iodine source, such as sodium iodide, in a suitable solvent such as acetonitrile.

Aryl bromides of formula (M) are commercially available or may be prepared by methods well known in the literature.

One skilled in the art will realise that it is often possible to alter the order in which the transformations described above are conducted, or to combine them in alternative ways to prepare a wide range of compounds of formula (I). Multiple steps may also be combined in a single reaction. All such variations are contemplated within the scope of the invention.

The skilled person will also be aware that some reagents will be incompatible with certain values or combinations of the substituents R¹, R², R³, R⁴, R⁵ and R⁶, as defined herein, and any additional steps, such as protection and/or deprotection steps, which are necessary to achieve the desired transformation will be clear to the skilled person.

The compounds according to the invention can be used as herbicidal agents in unmodified form, but they are generally formulated into compositions in various ways using formulation adjuvants, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO and WHO Specifications for Pesticides, United Nations, First Edition, Second Revision (2010). For water-soluble compounds, soluble liquids, water-soluble concentrates or water soluble granules are preferred. Such formulations can either be used directly or diluted prior to use. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof.

The active ingredients can also be contained in very fine microcapsules. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxypropanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances.

A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecyl-benzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in McCutcheon's Detergents and Emulsifiers Annual, MC Publishing Corp., Ridgewood New Jersey (1981).

Further adjuvants that can be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and liquid and solid fertilisers.

The compositions according to the invention can include an additive comprising an oil of vegetable or animal origin, a mineral oil, alkyl esters of such oils or mixtures of such oils and oil derivatives. The amount of oil additive in the composition according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C₈-C₂₂ fatty acids, especially the methyl derivatives of C₁₂-C₁₈ fatty acids, for example the methyl esters of lauric acid, palmitic acid and oleic acid (methyl laurate, methyl palmitate and methyl oleate, respectively). Many oil derivatives are known from the Compendium of Herbicide Adjuvants, 10^{th} Edition, Southern Illinois University, 2010.

The herbicidal compositions generally comprise from 0.1 to 99% by weight, especially from 0.1 to 95% by weight, compounds of formula (I) and from 1 to 99.9% by weight of a formulation adjuvant which preferably includes from 0 to 25% by weight of a surface-active substance. The inventive compositions generally comprise from 0.1 to 99% by weight, especially from 0.1 to 95% by weight, of compounds of the present invention and from 1 to 99.9% by weight of a formulation adjuvant which preferably includes from 0 to 25% by weight of a surface-active substance. Whereas commercial products may preferably be formulated as concentrates, the end user will normally employ dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline compounds may be applied at a rate of from 1 to 2000 I/ha, especially from 10 to 1000 I/ha.

Preferred formulations can have the following compositions (weight %):

### Emulsifiable Concentrates:

active ingredient: 1 to 95%, preferably 60 to 90%
surface-active agent: 1 to 30%, preferably 5 to 20%
liquid carrier: 1 to 80%, preferably 1 to 35%

### Dusts:

active ingredient: 0.1 to 10%, preferably 0.1 to 5%
solid carrier: 99.9 to 90%, preferably 99.9 to 99%

### Suspension Concentrates:

active ingredient: 5 to 75%, preferably 10 to 50%
water: 94 to 24%, preferably 88 to 30%
surface-active agent: 1 to 40%, preferably 2 to 30%

### Wettable Powders:

active ingredient: 0.5 to 90%, preferably 1 to 80%
surface-active agent: 0.5 to 20%, preferably 1 to 15%
solid carrier: 5 to 95%, preferably 15 to 90%

### Granules:

active ingredient: 0.1 to 30%, preferably 0.1 to 15%
solid carrier: 99.5 to 70%, preferably 97 to 85%

The composition of the present may further comprise at least one additional pesticide. For example, the compounds according to the invention can also be used in combination with other herbicides or plant growth regulators. In a preferred embodiment the additional pesticide is a herbicide and/or herbicide safener.

The compounds of present invention can also be used in mixture with one or more additional herbicides and/or plant growth regulators. Examples of such additional herbicides or plant growth regulators include acetochlor, acifluorfen (including acifluorfen-sodium), aclonifen, ametryn, amicarbazone, aminopyralid, aminotriazole, atrazine, beflubutamid-M, benquitrione, bensulfuron (including bensulfuron-methyl), bentazone, bicyclopyrone, bilanafos, bipyrazone, bispyribac-sodium, bixlozone, bromacil, bromoxynil, butachlor, butafenacil, carfentrazone (including carfentrazone-ethyl), cloransulam (including cloransulam-methyl), chlorimuron (including chlorimuron-ethyl), chlorotoluron, chlorsulfuron, cinmethylin, clacyfos, clethodim, clodinafop (including clodinafop-propargyl), clomazone, clopyralid, cyclopyranil, cyclopyrimorate, cyclosulfamuron, cyhalofop (including cyhalofop-butyl), 2,4-D (including the choline salt and 2-ethylhexyl ester thereof), 2,4-DB, desmedipham, dicamba (including the aluminium, aminopropyl, bis-aminopropylmethyl, choline, dichloroprop, diglycolamine, dimethylamine, dimethylammonium, potassium and sodium salts thereof) diclosulam, diflufenican, diflufenzopyr, dimethachlor, dimethenamid-P, dioxopyritrione, diquat dibromide, diuron, epyrifenacil, ethalfluralin, ethofumesate, fenoxaprop (including fenoxaprop-P-ethyl), fenoxasulfone, fenpyrazone, fenquinotrione, fentrazamide, flazasulfuron, florasulam, florpyrauxifen (including florpyrauxifen-benzyl), fluazifop (including fluazifop-P-butyl), flucarbazone (including flucarbazone-sodium), flufenacet, flumetsulam, flumioxazin, fluometuron, flupyrsulfuron (including flupyrsulfuron-methyl-sodium), fluroxypyr (including fluroxypyr-meptyl), fomesafen, foramsulfuron, glufosinate (including L-glufosinate and the ammonium salts of both), glyphosate (including the diammonium, isopropylammonium and potassium salts thereof), halauxifen (including halauxifen-methyl), haloxyfop (including haloxyfop-methyl), hexazinone, hydantocidin, imazamox (including R-imazamox), imazapic, imazapyr, imazethapyr, indaziflam, iodosulfuron (including iodosulfuron-methyl-sodium), iofensulfuron (including iofensulfuron-sodium), ioxynil, isoproturon, isoxaflutole, lancotrione, MCPA, MCPB, mecoprop-P, mesosulfuron (including mesosulfuron-methyl), mesotrione, metamitron, metazachlor, methiozolin, metolachlor, metosulam, metribuzin, metsulfuron, napropamide, nicosulfuron, norflurazon, oxadiazon, oxasulfuron, oxyfluorfen, paraquat dichloride, pendimethalin, penoxsulam, phenmedipham, picloram, pinoxaden, pretilachlor, primisulfuron-methyl, prometryne, propanil, propaquizafop, propyrisulfuron, propyzamide, prosulfocarb, prosulfuron, pyraclonil, pyraflufen (including pyraflufen-ethyl), pyrasulfotole, pyridate, pyriftalid, pyrimisulfan, pyroxasulfone, pyroxsulam, quinclorac, quinmerac, quizalofop (including quizalofop-P-ethyl and quizalofop-P-tefuryl), rimisoxafen, rimsulfuron, saflufenacil, sethoxydim, simazine, S-metalochlor, sulfentrazone, sulfosulfuron, tebuthiuron, tefuryltrione, tembotrione, terbuthylazine, terbutryn, tetflupyrolimet, thiencarbazone, thifensulfuron, tiafenacil, tolpyralate, topramezone, tralkoxydim, triafamone, triallate, triasulfuron, tribenuron (including tribenuron-methyl), triclopyr, trifloxysulfuron (including trifloxysulfuron-sodium), trifludimoxazin, trifluralin, triflusulfuron, tripyrasulfone, 3-(2-chloro-4-fluoro-5-(3-methyl-2,6-dioxo-4-trifluoromethyl-3,6-dihydropyrimidin-1(2H)-yl)phenyl)-5-methyl-4,5-dihydroisoxazole-5-carboxylic acid ethyl ester, 4-hydroxy-1-methoxy-5-methyl-3-[4-(trifluoromethyl)-2-pyridyl]imidazolidin-2-one, 4-hydroxy-1,5-dimethyl-3-[4-(trifluoromethyl)-2-pyridyl]imidazolidin-2-one, 5-ethoxy-4-hydroxy-1-methyl-3-[4-(trifluoromethyl)-2-pyridyl]imidazolidin-2-one, 4-hydroxy-1-methyl-3-[4-(trifluoromethyl)-2-pyridyl]imidazolidin-2-one, 4-hydroxy-1,5-dimethyl-3-[1-methyl-5-(trifluoromethyl)pyrazol-3-yl]imidazolidin-2-one, (4R)1-(5-tert-butylisoxazol-3-yl)-4-ethoxy-5-hydroxy-3-methyl-imidazolidin-2-one, 4-amino-3-chloro-5-fluoro-6-(7-fluoro-1H-indol-6-yl)pyridine-2-carboxylic acid (including agrochemically acceptable esters thereof, for example, methyl 4-amino-3-chloro-5-fluoro-6-(7-fluoro-1H-indol-6-yl)pyridine-2-carboxylate, prop-2-ynyl 4-amino-3-chloro-5-fluoro-6-(7-fluoro-1H-indol-6-yl)pyridine-2-carboxylate and cyanomethyl 4-amino-3-chloro-5-fluoro-6-(7-fluoro-1H-indol-6-yl)pyridine-2-carboxylate), 3-ethylsulfanyl-N-(1,3,4-oxadiazol-2-yl)-5-(trifluoromethyl)-[1,2,4]triazolo[4,3-a]pyridine-8-carboxamide, 3-(isopropylsulfanylmethyl)-N-(5-methyl-1,3,4-oxadiazol-2-yl)-5-(trifluoromethyl)-[1,2,4]triazolo[4,3-a]pyridine-8-carboxamide, 3-(isopropylsulfonylmethyl)-N-(5-methyl-1,3,4-oxadiazol-2-yl)-5-(trifluoromethyl)-[1,2,4]triazolo[4,3-a]pyridine-8-carboxamide, 3-(ethylsulfonylmethyl)-N-(5-methyl-1,3,4-oxadiazol-2-yl)-5-(trifluoromethyl)-[1,2,4]triazolo[4,3-a]pyridine-8-carboxamide, ethyl 2-[[3-[[3-chloro-5-fluoro-6-[3-methyl-2,6-dioxo-4-(trifluoromethyl)pyrimidin-1-yl]-2-pyridyl]oxy]acetate and 6-chloro-4-(2,7-dimethyl-1-naphthyl)-5-hydroxy-2-methyl-pyridazin-3-one.

The mixing partners of the compound of formula (I) may also be in the form of esters or salts, as mentioned e.g. in The Pesticide Manual, Fourteenth Edition, British Crop Protection Council, 2006.

The compound of formula (I) can also be used in mixtures with other agrochemicals such as fungicides, nematicides or insecticides, examples of which are given in The Pesticide Manual.

The mixing ratio of the compound of formula (I) to the mixing partner is preferably from 1:100 to 1000:1.

The mixtures can advantageously be used in the above-mentioned formulations (in which case “active ingredient” relates to the respective mixture of compound of formula (I) with the mixing partner).

Compounds of formula (I) of the present invention may also be combined with herbicide safeners. Examples of such safeners include benoxacor, cloquintocet (including cloquintocetmexyl), cyprosulfamide, dichlormid, fenchlorazole (including fenchloraz oleethyl), fenclorim, fluxofenim, furilazole, isoxadifen (including isoxadifenethyl), mefenpr (including mefenpyr-diethyl), metcamifen and oxabetrinil.

Particularly preferred are mixtures of a compound of formula (I) with cyprosulfamide, isoxadifen (including isoxadifen-ethyl), cloquintocet (including cloquintocet-mexyl) and/or N-(2-methoxybenzoyl)-4-[(methyl-aminocarbonyl)amino]benzenesulfonamide.

The safeners of the compound of formula (I) may also be in the form of esters or salts, as mentioned e.g. in The Pesticide Manual, 14^{th} Edition (BCPC), 2006. The reference to cloquintocet-mexyl also applies to a lithium, sodium, potassium, calcium, magnesium, aluminium, iron, ammonium, quaternary ammonium, sulfonium or phosphonium salt thereof as disclosed in WO 02/34048, and the reference to fenchlorazole-ethyl also applies to fenchlorazole, etc.

Preferably the mixing ratio of compound of formula (I) to safener is from 100:1 to 1:10, especially from 20:1 to 1:1.

The mixtures can advantageously be used in the above-mentioned formulations (in which case “active ingredient” relates to the respective mixture of compound of formula (I) with the safener).

The compounds of formula (I) of this invention are useful as herbicides. The present invention therefore further comprises a method for controlling unwanted plants comprising applying to the said plants or a locus comprising them, an effective amount of a compound of the invention or a herbicidal composition containing said compound. ‘Controlling’ means killing, reducing or retarding growth or preventing or reducing germination. Generally the plants to be controlled are unwanted plants (weeds). ‘Locus’ means the area in which the plants are growing or will grow.

The rates of application of compounds of formula (I) may vary within wide limits and depend on the nature of the soil, the method of application (pre-emergence; post-emergence; application to the seed furrow; no tillage application etc.), the crop plant, the weed(s) to be controlled, the prevailing climatic conditions, and other factors governed by the method of application, the time of application and the target crop. The compounds of formula (I) according to the invention are generally applied at a rate of from 10 to 2000 g/ha, especially from 50 to 1000 g/ha. A preferred range is 10-200 g/ha.

The application is generally made by spraying the composition, typically by tractor mounted sprayer for large areas, but other methods such as dusting (for powders), drip or drench can also be used.

Useful plants in which the composition according to the invention can be used include crops such as cereals, for example barley and wheat, cotton, oilseed rape, sunflower, maize, rice, soybeans, sugar beet, sugar cane and turf.

Crop plants can also include trees, such as fruit trees, palm trees, coconut trees or other nuts. Also included are vines such as grapes, fruit bushes, fruit plants and vegetables.

Crops are to be understood as also including those crops which have been rendered tolerant to herbicides or classes of herbicides (e.g. ALS-, GS-, EPSPS-, PPO-, ACCase- and HPPD-inhibitors) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®.

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of toxins, or transgenic plants able to synthesise such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crops are also to be understood to include those which are obtained by conventional methods of breeding or genetic engineering and contain so-called output traits (e.g. improved storage stability, higher nutritional value and improved flavour).

Other useful plants include turf grass for example in golf-courses, lawns, parks and roadsides, or grown commercially for sod, and ornamental plants such as flowers or bushes.

Compounds of formula (I) and compositions of the invention can typically be used to control a wide variety of monocotyledonous and dicotyledonous weed species. Examples of monocotyledonous species that can typically be controlled include Alopecurus myosuroides, Avena fatua, Brachiaria plantaginea, Bromus tectorum, Cyperus esculentus, Digitaria sanguinalis, Echinochloa crus-galli, Lolium perenne, Lolium multiflorum, Panicum miliaceum, Poa annua, Setaria viridis, Setaria faberi and Sorghum bicolor. Examples of dicotyledonous species that can be controlled include Abutilon theophrasti, Amaranthus retroflexus, Bidens pilosa, Chenopodium album, Euphorbia heterophylla, Galium aparine, Ipomoea hederacea, Kochia scoparia, Polygonum convolvulus, Sida spinosa, Sinapis arvensis, Solanum nigrum, Stellaria media, Veronica persica and Xanthium strumarium.

The compounds of formula (I) are also useful for pre-harvest desiccation in crops, for example, but not limited to, potatoes, soybean, sunflowers and cotton. Pre-harvest desiccation is used to desiccate crop foliage without significant damage to the crop itself to aid harvesting.

Compounds/compositions of the invention are particularly useful in non-selective burn-down applications, and as such may also be used to control volunteer or escape crop plants.

Various aspects and embodiments of the present invention will now be illustrated in more detail by way of example. It will be appreciated that modification of detail may be made without departing from the scope of the invention.

### EXAMPLES

The Examples which follow serve to illustrate, but do not limit, the invention.

### Synthesis Examples

### Example 1 Preparation of 2-(8-chloro-1-naphthyl)-2,2-difluoro-acetic acid (Compound 1-416)

### Step 1 Synthesis of ethyl 2-(8-chloro-1-naphthyl)-2,2-difluoro-acetate (Compound 3-416)

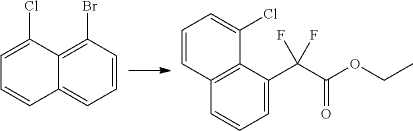 [SMILES: Clc1cccc2cccc(Br)c12 ; CCOC(=O)C(F)(F)c1cccc2cccc(Cl)c12]

To a solution of 1-bromo-8-chloro-naphthalene (1.00 g, 4.14 mmol) in DMSO (5 mL) was added Cu powder (1.32 g, 20.7 mmol) followed by ethyl 2,2-difluoro-2-iodo-acetate (1.24 g, 4.97 mmol). The reaction mixture was stirred at room temperature for 48 h. The reaction mixture was diluted with water (20 mL) and extracted with ethyl acetate (3×30 mL). The combined organic layers were washed with brine (30 mL), dried over Na₂SO₄ and concentrated under reduced pressure. The crude product was purified by column chromatography (2% EtOAc in n-hexane) to give ethyl 2-(8-chloro-1-naphthyl)-2,2-difluoro-acetate (0.200 g, 0.632 mmol, 15%) as a colourless oil.

¹H NMR (400 MHz, CDCl₃): δ 8.10 (d, 1H), 7.97 (d, 1H), 7.81 (dd, 1H), 7.62 (dd, 1H), 7.53 (t, 1H), 7.38 (t, 1H), 4.36 (q, 2H), 1.30 (t, 3H) ppm

Also prepared by this general method were:

Methyl 2-(3,7-dichloro-8-quinolyl)-2,2-difluoro-acetate (Compound 2-418)

¹H NMR (400 MHz, CDCl₃): δ 8.73 (s, 1H), 8.14 (s, 1H), 7.80-7.78 (d, 1H), 7.65-7.63 (m, 1H), 3.85 (s, 3H) ppm

Ethyl 2-[8-(2-ethoxy-1,1-difluoro-2-oxo-ethyl)-1-naphthyl]-2,2-difluoro-acetate (Compound 3-419)

¹H NMR (400 MHz, CDCl3): δ 8.11 (d, 1H), 7.99 (d, 1H), 7.90 (dt, 2H), 7.56 (t, 1H), 7.33 (t, 1H), 4.38 (q, 4H), 1.33 (t, 6H) ppm

Ethyl 2-(2-chloro-6-nitrophenyl)-2,2-difluoroacetate (Compound 3-67)

¹H NMR (400 MHz, DMSO-d6): δ 8.03-7.99 (m, 2H), 7.91-7.87 (m, 1H), 4.40 (q, 2H), 1.25 (t, 3H) ppm

### Step 2 Synthesis of 2-(8-chloro-1-naphthyl)-2,2-difluoro-acetic acid (Compound 1-416)

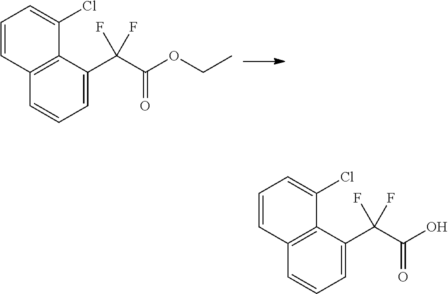 [SMILES: CCOC(=O)C(F)(F)c1cccc2cccc(Cl)c12 ; O=C(O)C(F)(F)c1cccc2cccc(Cl)c12]

To a solution of ethyl 2-(8-chloro-1-naphthyl)-2,2-difluoro-acetate (200 mg, 0.632 mmol) in 2:1 THE/Water (3.0 mL) was added LiOH·H2O (0.0531 g, 1.26 mmol) portion-wise and the reaction mixture was stirred at room temperature for 16 h. The reaction mixture was concentrated and diluted with water (20 mL) and extracted with ethyl acetate (3×30 mL). The combined organic layers were washed with 1N HCl solution (30 mL) and dried over Na₂SO₄ and concentrated under reduced pressure. The crude product was purified by reverse phase column chromatography (product eluted at 0-50% Acetonitrile in water) to give 2-(8-chloro-1-naphthyl)-2,2-difluoro-acetic acid (0.0550 g, 0.212 mmol, 34%) as an off-white solid.

¹H NMR (400 MHz, DMSO-d6): δ 14.91-14.56 (br s, 1H), 8.24 (d, 1H), 8.10 (m, 2H), 7.79 (d, 1H), 7.70 (t, 1H), 7.59 (t, 1H) ppm

Also prepared by this general method were:

Ammonium 2-(3-chloro-2-nitro-phenyl)-2,2-difluoro-acetate (Compound 7-250)

¹H NMR (400 MHz, DMSO-d6) δ 7.84 (t, 1H), 7.70-7.65 (m, 2H), 7.19 (br s, 4H) ppm

2-(3,7-Dichloro-8-quinolyl)-2,2-difluoro-acetic acid (Compound 1-418)

¹H NMR (400 MHz, DMSO-d6) δ 14.7-14.2 (br s, 1H), 8.93-8.92 (d, 1H), 8.74-8.73 (d, 1H), 8.19-8.17 (d, 1H), 7.85-7.83 (d, 1H) ppm

2-(3-Aminophenyl)-2,2-difluoro-acetic acid (Compound 1-8)

¹H NMR (400 MHz, DMSO-d6) δ 6.98 (t, 1H), 6.74 (s, 1H), 6.63 (d, 1H), 6.52 (d, 1H), 5.11 (s, 2H) ppm

2-(3-Chloro-2-cyano-phenyl)-2,2-difluoro-acetic acid (Compound 1-262)

¹H NMR (400 MHz, DMSO-d6) δ 8.00 (d, 1H), 7.90 (t, 1H), 7.81 (d, 1H) ppm

2-(2-Chloro-3-ethynyl-phenyl)-2,2-difluoro-acetic acid (Compound 1-208)

¹H NMR (400 MHz, DMSO-d6) δ 7.66-7.60 (m, 2H), 7.39 (t, 1H), 4.58 (s, 1H) ppm

2,2-Difluoro-2-(1-naphthyl)acetic acid (Compound 1-397)

¹H NMR (400 MHz, DMSO-d6) δ 8.16 (d, 1H), 8.13-8.02 (m, 2H), 7.86 (d, 1H), 7.75-7.55 (m, 3H) ppm

2,2-Difluoro-2-(quinoline-8-yl)acetic acid (Compound 1-403)

¹H NMR (400 MHz, DMSO-d6): δ 8.91 (dd, 1H), 8.49 (dd, 1H), 8.20 (d, 1H), 8.11 (d, 1H), 7.74 (t, 1H), 7.64 (dd, 1H) ppm

2,2-Difluoro-2-(3-hydroxyphenyl)acetic acid (Compound 1-5)

¹H NMR (400 MHz, DMSO-d6): δ 9.87 (br s, 1H), 7.32 (t, 1H), 7.0-6.89 (m, 3H) ppm

2-(2-Chloro-6-nitrophenyl)-2,2-difluoroacetic acid (Compound 1-67)

¹H NMR (400 MHz, DMSO-d6): δ 7.96-7.93 (m, 2H), 7.85-7.81 (m, 1H) ppm

2-(7-Chloroquinolin-8-yl)-2,2-difluoroacetic acid (Compound 1-407)

¹H-NMR (400 MHz, DMSO-d6): δ 14.15 (brs, 1H), 8.91-8.89 (m, 1H), 8.52-8.50 (m, 1H), 8.21 (d, 1H), 7.68-7.65 (m, 2H) ppm

2,2-Difluoro-2-(2,3,5-trichloro-6-methoxy-phenyl)acetic acid (Compound 1-82)

¹H NMR (400 MHz, DMSO-d6): δ 8.17 (s, 1H), 3.78 (s, 3H) ppm

2-(2,5-Dichloro-3-nitrophenyl)-2,2-difluoroaceticacid (Compound 1-238)

¹H NMR (400 MHz, DMSO-d6): δ 8.32 (d, 1H), 7.84 (d, 1H) ppm

2-(3-Amino-2,5-dichlorophenyl)-2,2-difluoroacetic acid (Compound 1-235)

¹H NMR (400 MHz, DMSO-d6): δ 6.99 (d, 1H), 6.82 (d, 1H), 5.99 (brs, 2H) ppm

2-(3-Chloro-2-(methylthio)phenyl)-2,2-difluoroacetic acid (Compound 1-356)

¹H NMR (400 MHz, DMSO-d6): δ 7.81 (d, 1H), 7.71 (dd, 1H), 7.58 (t, 1H), 2.29 (s, 3H) ppm

2-(2-Chloro-3-methylsulfanyl-phenyl)-2,2-difluoro-acetic acid (Compound 1-220)

¹H NMR (400 MHz, DMSO-d6): δ 7.55-7.48 (m, 3H), 2.54 (s, 3H) ppm

2-(3-Amino-2,6-dichloro-phenyl)-2,2-difluoro-acetic acid (Compound 1-236)

¹H NMR (400 MHz, DMSO-d6): δ 7.2 (d, 1H), 6.93 (d, 1H), 5.87 (br s, 2H) ppm

2-(5-Chloro-2,2-difluorobenzo[d][1,3]dioxol-4-yl)-2,2-difluoroaceticacid (Compound 1-389)

¹H NMR (400 MHz, DMSO-d6): δ 7.45 (d, 1H), 7.30 (d, 1H) ppm

2-(4-Chloro-4′-fluoro-[1,1′-biphenyl]-3-yl)-2,2-difluoroacetic acid (Compound 1-64)

¹H NMR (400 MHz, DMSO-d6): δ 7.92 (d, 1H), 7.86 (dd, 1H), 7.78 (m, 2H), 7.69 (d, 1H), 7.30 (t, 2H) ppm

2-(4,5-Dichloro-4′-fluoro-[1,1′-biphenyl]-3-yl)-2,2-difluoroacetic acid (Compound 1-78)

¹H NMR (400 MHz, DMSO-d6): δ 7.98-7.97 (d, 1H), 7.79-7.76 (m, 2H), 7.73 (d, 1H), 7.34-7.30 (m, 2H) ppm

2,2-Difluoro-2-(quinoxalin-5-yl)acetic acid (Compound 1-409)

¹H NMR (400 MHz, DMSO-d6): δ 14.50 (s, 1H), 9.06 (d, 1H), 8.99 (d, 1H), 8.32 (d, 1H), 8.22 (d, 1H), 8.01 (t, 1H) ppm

2-(2-Chloro-3-ethyl-phenyl)-2,2-difluoro-acetic acid (Compound 1-196)

¹H NMR (400 MHz, CDCl3): δ 7.59 (d, 1H), 7.39 (d, 1H), 7.31 (d, 1H), 2.76-2.81 (q, 2H), 1.24 (t, 3H) ppm

2-(3-Chloro-2-ethynyl-phenyl)-2,2-difluoro-acetic acid (Compound 1-291)

¹H NMR (400 MHz, DMSO-d6): δ 7.78 (d, 1H), 7.68 (dd, 1H), 7.58 (t, 1H), 4.98 (s, 1H) ppm

2-(2-Chloro-3-vinyl-phenyl)-2,2-difluoro-acetic acid (Compound 1-207)

¹H NMR (400 MHz, DMSO-d6): δ 7.92 (dd, 1H), 7.70 (dd, 1H), 7.52 (t, 1H), 7.06 (m, 1H), 5.96 (dd, 1H), 5.55 (dd, 1H) ppm

2-(3-Chloro-2-ethyl-phenyl)-2,2-difluoro-acetic acid (Compound 1-275)

¹H NMR (400 MHz, DMSO-d6): δ 7.42 (t, 1H), 7.23 (t, 1H), 6.09 (s, 1H), 2.88-2.82 (q, 2H), 1.07 (t, 3H) ppm

2-(2-Chloro-5-hydroxyphenyl)-2,2-difluoroacetic acid (Compound 1-61)

¹H NMR (400 MHz, DMSO-d6): δ 10.1 (brs, 1H), 7.36 (d, 1H), 7.09 (s, 1H), 6.93 (dd, 1H) ppm

Ammonium 2-(2-chloro-3-nitro-phenyl)-2,2-difluoro-acetate (Compound 7-237)

¹H NMR (400 MHz, DMSO-d6): δ 7.9 (d, 1H), 7.71 (dd, 1H), 7.51 (t, 1H) ppm

Ammonium;2-(2-chloro-3-cyano-phenyl)-2,2-difluoro-acetate (Compound 7-231)

¹H NMR (400 MHz, DMSO-d6): δ 8.01 (d, 1H), 7.91 (d, 1H), 7.6 (t, 1H) ppm

2-(4-Bromo-3-chloro-2-methylsulfanyl-phenyl)-2,2-difluoro-acetic acid (Compound 1-359)

¹H-NMR (400 MHz, DMSO-d6): δ 7.59 (d, 1H), 7.39 (d, 1H), 2.67 (s, 3H) ppm

2-(2-Chloro-5-methoxyphenyl)-2,2-difluoroacetic acid (Compound 1-62)

¹H NMR (400 MHz, DMSO-d6): δ 14.68 (br s, 1H), 7.61 (dd, 1H), 7.55 (d, 1H), 7.22 (d, 1H), 3.71 (s, 3H) ppm

2,2-Difluoro-2-(2-fluoro-3-methoxy-phenyl)acetic acid (Compound 1-190)

¹H NMR (500 MHz, chloroform) δ=7.34 (br s, 1H), 7.23-7.13 (m, 2H), 7.12-7.05 (m, 1H), 3.90 (s, 3H) ppm

2-(3-Cyano-2-fluoro-phenyl)-2,2-difluoro-acetic acid (Compound 1-192)

¹H NMR (500 MHz, chloroform) δ=8.02-7.89 (m, 1H), 7.84 (br s, 1H), 7.79 (br t, 1H), 7.40 (t, 1H) ppm

2,2-Difluoro-2-(3-methoxy-2-methyl-phenyl)acetic acid (Compound 1-242)

¹H NMR (500 MHz, chloroform) δ=7.71 (br s, 1H), 7.25-7.20 (m, 2H), 6.95 (dd, 1H), 3.83 (s, 3H), 2.27 (s, 3H) ppm

2-(3-Cyano-2-methyl-phenyl)-2,2-difluoro-acetic acid (Compound 1-244)

¹H NMR (500 MHz, chloroform) δ 8.38 (br s, 1H), 7.85 (d, 1H), 7.73 (d, 1H), 7.41 (t, 1H), 2.64 (s, 3H) ppm

2-(2-Cyano-3-fluoro-phenyl)-2,2-difluoro-acetic acid (Compound 1-261)

¹H NMR (500 MHz, chloroform) δ=7.92-7.53 (m, 3H), 7.52-7.32 (m, 1H) ppm

2-(2,3-Dihydrobenzofuran-4-yl)-2,2-difluoro-acetic acid (Compound 1-420)

¹H NMR (500 MHz, chloroform) δ=7.23-7.14 (m, 1H), 7.06 (d, 1H), 6.89 (d, 1H), 6.84 (br s, 1H), 4.58 (t, 2H), 3.40 (t, 2H) ppm

2-(3-Chloro-2-cyclopropyl-phenyl)-2,2-difluoro-acetic acid (Compound 1-428)

¹H NMR (400 MHz, chloroform) δ=7.60 (dd, 1H) 7.51 (d, 1H) 7.26-7.32 (m, 1H) 1.77-1.87 (m, 1H) 1.03-1.10 (m, 2H) 0.77 (q, 2H) ppm

2-[3-Chloro-2-(methoxymethyl)phenyl]-2,2-difluoro-acetic acid (Compound 1-436)

¹H NMR (400 MHz, chloroform) δ=7.53-7.62 (m, 2H) 7.35-7.41 (m, 1H) 4.93 (s, 2H) 3.47 (s, 3H) ppm

2-(3-Chloro-2-methoxycarbonyl-phenyl)-2,2-difluoro-acetic acid (Compound 1-444)

¹H NMR (500 MHz, chloroform) δ=8.17 (br s, 1H), 7.62 (d, 1H), 7.54 (d, 1H), 7.49-7.41 (m, 1H), 3.92 (s, 3H) ppm

2-[3-Chloro-2-(difluoromethoxy)phenyl]-2,2-difluoro-acetic acid (Compound 1-452)

¹H NMR (500 MHz, chloroform) δ=7.67 (dd, 1H), 7.60 (d, 1H), 7.51 (br s, 1H), 7.33 (t, 1H), 6.64 (t, 1H) ppm

2-(2,4-Difluoro-3-methoxy-phenyl)-2,2-difluoro-acetic acid (Compound 1-458)

¹H NMR (500 MHz, chloroform) δ=7.56 (br s, 1H), 7.35-7.22 (m, 1H), 7.05-6.90 (m, 1H), 4.01 (s, 3H) ppm

2-[2-Chloro-3-(difluoromethoxy)phenyl]-2,2-difluoro-acetic acid (Compound 1-459)

¹H NMR (400 MHz, chloroform) δ=9.27 (br s, 1H) 7.35-7.74 (m, 3H) 6.34-6.80 (m, 1H) ppm

2-(2-Chloro-3-cyano-4-fluoro-phenyl)-2,2-difluoro-acetic acid (Compound 1-460)

¹H NMR (500 MHz, chloroform) δ=7.99 (dd, 1H), 7.62 (br s, 1H), 7.35-7.23 (m, 1H) ppm

2,2-Difluoro-2-(3-fluoro-4-methoxy-2-nitro-phenyl)acetic acid (Compound 1-461)

¹H NMR (500 MHz, chloroform) δ=7.60 (br s, 1H), 7.53 (dd, 1H), 7.18 (t, 1H), 3.99 (s, 3H) ppm

2-(2-Cyano-3-methyl-phenyl)-2,2-difluoro-acetic acid (Compound 1-462)

¹H NMR (500 MHz, chloroform) δ=7.63-7.51 (m, 2H), 7.47 (d, 1H), 7.13 (br s, 1H), 2.61 (s, 3H) ppm

2,2-Difluoro-2-(2-methoxy-3-methyl-phenyl)acetic acid (Compound 1-463)

¹H NMR (500 MHz, chloroform) δ=7.78 (br s, 1H), 7.49 (d, 1H), 7.31 (d, 1H), 7.12 (t, 1H), 3.78 (s, 3H), 2.31 (s, 3H) ppm

2,2-Difluoro-2-[2-methoxy-3-(trifluoromethyl)phenyl]acetic acid (Compound 1-464)

¹H NMR (500 MHz, chloroform) δ=7.90 (d, 1H), 7.76 (d, 1H), 7.35 (t, 1H), 3.91 (s, 3H) ppm

2-[2-Chloro-4-(trifluoromethoxy)phenyl]-2,2-difluoro-acetic acid (Compound 1-59)

¹H NMR (500 MHz, chloroform) δ=7.78 (d, 1H), 7.32 (s, 1H), 7.24 (d, 1H) ppm

2,2-Difluoro-2-[2-(trifluoromethoxy)phenyl]acetic acid (Compound 1-465)

¹H NMR (500 MHz, chloroform) δ=7.76 (d, 1H), 7.57-7.51 (m, 1H), 7.38 (t, 1H), 7.34 (d, 1H) ppm

2-(4-Cyanophenyl)-2,2-difluoro-acetic acid (Compound 1-474)

¹H NMR (400 MHz, DMSO-d6) δ=8.00 (d, 2H), 7.76 (d, 2H)

2,2-Difluoro-2-(1H-indol-7-yl)acetic acid (Compound 1-477)

¹H NMR (400 MHz, DMSO-d6) δ=11.12 (br s, 1H), 7.59 (d, 1H), 7.36 (d, 1H), 7.21 (d, 1H), 7.00 (t, 1H), 6.44 (d, 1H) ppm

### Example 2 Preparation of 2-(3-chloro-2-methoxy-phenyl)-2,2-difluoro-acetic acid (Compound 1-317)

### Step 1 Synthesis of ethyl 2-(3-chloro-2-methoxy-phenyl)-2-oxo-acetate

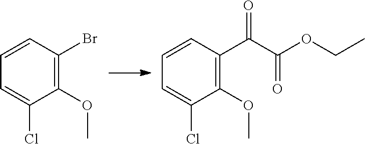

To a stirred solution of 1-bromo-3-chloro-2-methoxy-benzene (1.00 g, 4.52 mmol) in tetrahydrofuran (20 mL) at −78° C. was added n-BuLi (0.289 g, 4.52 mmol) and the reaction was stirred at −78° C. for 15 minutes. Diethyl oxalate (0.660 g, 4.52 mmol) was added and the reaction was stirred at −78° C. for 2 hours. The reaction mixture was warmed to room temperature and quenched with ammonium chloride solution (20 mL) and extracted with ethyl acetate (30 mL). The organic layer was washed with brine solution (10 mL) and dried over Na₂SO₄ and concentrated under reduced pressure to afford crude ethyl 2-(3-chloro-2-methoxy-phenyl)-2-oxo-acetate (0.600 g, 2.23 mmol, 49%) as a colorless oil.

¹H NMR (400 MHz, DMSO-d6): δ 7.92 (d, 1H), 7.78 (d, 1H), 7.39 (t, 1H), 4.38 (q, 2H), 3.83 (s, 3H), 1.31 (t, 3H) ppm

Also prepared by this general method were:

Ethyl 2-(7-fluoro-8-quinolyl)-2-oxo-acetate

¹H NMR (400 MHz, CDCl3): δ 9.08 (m, 1H), 8.87 (dd, 1H), 8.02 (dd, 1H), 7.80 (dd, 1H), 7.46 (dd, 1H), 4.32 (q, 2H), 1.37 (t, 3H) ppm

Ethyl 2-(2,2-Difluoro-1,3-benzodioxol-4-yl)-2-oxo-acetate

¹H NMR (400 MHz, CDCl3): δ 7.66 (d, 1H), 7.33 (d, 1H), 7.21 (m, 1H), 4.46 (q, 2H), 1.41 (t, 3H) ppm

### Step 2 Synthesis of ethyl 2-(3-chloro-2-methoxy-phenyl)-2,2-difluoro-acetate (Compound 3-317)

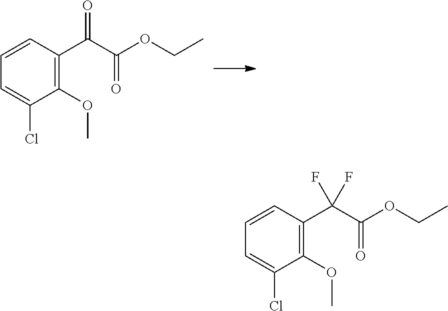 [SMILES: CCOC(=O)C(=O)c1cccc(Cl)c1OC ; CCOC(=O)C(F)(F)c1cccc(Cl)c1OC]

To a stirred solution of ethyl 2-(3-chloro-2-methoxy-phenyl)-2-oxo-acetate (680 mg, 2.52 mmol) in dichloromethane (10 mL) at 0° C. was added diethylaminosulfur trifluoride (0.813 g, 5.04 mmol). The resulting reaction mixture was stirred at room temperature for 16 hours. The reaction mixture was diluted with sodium bicarbonate solution and extracted with dichloromethane. The organic layer was dried over Na₂SO₄ and concentrated under reduced pressure. The resultant crude product was purified by column chromatography (compound eluted at 5-10% EtOAc in n-hexane) to give ethyl 2-(3-chloro-2-methoxy-phenyl)-2,2-difluoro-acetate (0.300 g, 1.02 mmol, 40%) as a colorless oil.

¹H NMR (400 MHz, CDCl3): δ 7.57 (dd, 1H), 7.51 (dd, 1H), 7.17 (t, 1H), 4.34 (q, 2H), 3.88 (s, 3H), 1.30 (t, 3H) ppm

Also prepared by this general method were:

Ethyl 2,2-difluoro-2-(7-fluoro-8-quinolyl)acetate (Compound 3-406)

¹H NMR (400 MHz, DMSO-d6): δ 8.92 (m, 1H), 8.57 (dd, 1H), 8.41 (dd, 1H), 7.76 (t, 1H), 7.67 (dd, 1H), 4.31 (q, 2H), 1.22 (t, 3H) ppm

Ethyl 2-(2,2-Difluoro-1,3-benzodioxol-4-yl)-2,2-difluoro-acetate (Compound 3-385)

### Step 3 Synthesis of 2-(3-chloro-2-methoxy-phenyl)-2,2-difluoro-acetic acid (Compound 1-317)

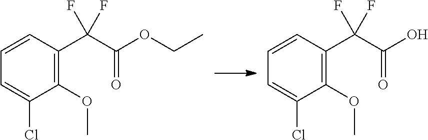 [SMILES: CCOC(=O)C(F)(F)c1cccc(Cl)c1OC ; COc1c(Cl)cccc1C(F)(F)C(=O)O]

To a solution of ethyl 2-(3-chloro-2-methoxy-phenyl)-2,2-difluoro-acetate (220 mg, 0.83 mmol) in 1:1 THE/Water (10 mL) was added lithium hydroxide monohydrate (70 mg, 1.66 mmol) and the reaction mixture was stirred at room temperature for 1 hour. The reaction mixture was treated with 2N HCl solution to bring the pH to 3 and filtered. The solid was dried to provide 2-(3-chloro-2-methoxy-phenyl)-2,2-difluoro-acetic acid (170 mg as an off-white solid.

¹H NMR (400 MHz, DMSO-d6): δ 14.7 (br s, 1H), 7.72 (dd, 1H), 7.70 (dd, 1H), 7.32 (t, 1H), 3.80 (s, 3H) ppm

Also prepared by this general method were:

2,2-Difluoro-2-(7-fluoro-8-quinolyl)acetic acid (Compound 1-406)

¹H NMR (400 MHz, DMSO-d6): δ 14.57-13.99 (br s, 1H), 8.92 (d, 1H), 8.51 (d, 1H), 8.31 (d, 1H), 7.81-7.48 (m, 2H) ppm

2-(2,2-Difluoro-1,3-benzodioxol-4-yl)-2,2-difluoro-acetic acid (Compound 1-385)

¹H NMR (400 MHz, DMSO-d6): δ 7.49 (d, 1H), 7.32-7.23 (m, 2H) ppm

2-(2-Chloro-3-methoxyphenyl)-2,2-difluoroacetic acid (Compound 1-209)

¹H NMR (400 MHz, DMSO-d6): δ 7.48 (t, 1H), 7.36 (d, 1H), 7.31 (dd, 1H), 3.9 (s, 3H) ppm

### Example 3 Preparation of 2-(3,6-dichloro-2-methoxy-phenyl)-2,2-difluoro-acetic acid (Compound 1-326)

### Step 1 Synthesis of 2-(3,6-dichloro-2-methoxy-phenyl)acetic acid

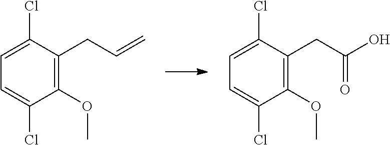

Ruthenium(III) chloride (0.73 g, 3.5 mmol) was added to a solution of 2-allyl-1,4-dichloro-3-methoxy-benzene (38 g, 180 mmol) in a mixture of water (530 mL), acetonitrile (350 mL) and ethyl acetate (350 mL). Sodium periodate (190 g, 880 mmol) was added portionwise over a period of 30 minutes keeping the internal temperature below 25° C. The mixture was stirred for 30 minutes. A solution of sodium metabisulfite (330 g, 1800 mmol) in water (500 mL) was prepared. The reaction mixture was cooled to 5° C. The solution of sodium metabisulfite was added to the reaction mixture at such a rate over 2 hours as to keep the internal temperature below 20° C. After the addition the starch-iodide paper test for oxidants was negative. The mixture was diluted with brine (400 mL) then separated. The aqueous layer was extracted with EtOAc (3×400 mL). The combined organic extracts were dried over MgSO4, filtered and concentrated in vacuo to provide a black oil. The crude product was purified by flash column chromatography to provide 2-(3,6-dichloro-2-methoxy-phenyl)acetic acid (30.59 g, 130.1 mmol, 74%) as an orange solid.

¹H NMR (400 MHz, CDCl3): δ 7.28 (d, 1H), 7.14 (d, 1H), 3.93 (s, 2H), 3.88 (s, 3H) ppm

### Step 2 Synthesis of methyl 2-(3,6-dichloro-2-methoxy-phenyl)acetate

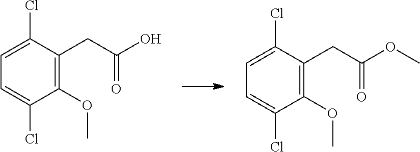

To 2-(3,6-dichloro-2-methoxy-phenyl)acetic acid (5.00 g, 21.3 mmol) in dichloromethane (60 mL) was added oxalyl chloride (2.74 mL, 31.9 mmol) followed by a catalytic amount of dimethylformamide. The reaction mixture was stirred at room temperature. After 2 hrs the reaction mixture was quenched carefully with methanol, diluted with water and extracted with ethyl acetate. The combined organic layers were dried over sodium sulfate and evaporated under reduced pressure. The crude material was purified by flash column chromatography to give methyl 2-(3,6-dichloro-2-methoxy-phenyl)acetate (5.27 g, 21.2 mmol, 99%).

¹H NMR (400 MHz, CDCl3): δ 7.26-7.24 (d, 1H), 7.12-7.10 (d, 1H), 3.86 (s, 2H), 3.84 (s, 3H), 3.71 (s, 3H) ppm

### Step 3 Synthesis of methyl 2-(3,6-dichloro-2-methoxy-phenyl)-2,2-difluoro-acetate (Compound 2-326)

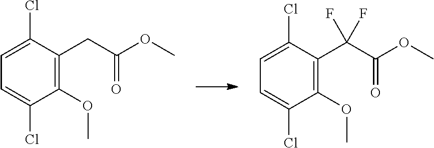 [SMILES: COC(=O)Cc1c(Cl)ccc(Cl)c1OC ; COC(=O)C(F)(F)c1c(Cl)ccc(Cl)c1OC]

To a solution of methyl 2-(3,6-dichloro-2-methoxy-phenyl)acetate (3.00 g, 12.0 mmol) in dry tetrahydrofuran (150 mL) was added potassium bis(trimethylsilyl)amide (0.50 M, 72.3 mL, 36.1 mmol) at −78° C. followed by the addition of N-fluorobenzenesulfonimide (12.2 g, 38.5 mmol). The reaction mixture was stirred overnight at room temperature. The reaction was quenched with saturated aqueous ammonium chloride and extracted with ethyl acetate. The combined organic layers were dried over sodium sulfate and evaporated under reduced pressure. The crude material was purified by flash column chromatography to give methyl 2-(3,6-dichloro-2-methoxy-phenyl)-2,2-difluoro-acetate (1.97 g, 6.91 mmol, 58%).

¹H NMR (400 MHz, CDCl3): δ 7.42-7.40 (d, 1H), 7.21-7.18 (d, 1H), 3.87 (s, 3H), 3.82 (s, 3H) ppm

Also prepared by this method was:

Methyl 2-(2-chloro-6-methoxy-phenyl)-2,2-difluoro-acetate (Compound 2-65)

¹H NMR (400 MHz, CDCl3): δ 7.34-7.30 (t, 1H), 7.09-7.07 (d, 1H), 6.86-6.84 (d, 1H), 3.87 (s, 3H), 3.79 (s, 3H) ppm

### Step 4 Synthesis of 2-(3,6-dichloro-2-methoxy-phenyl)-2,2-difluoro-acetic acid

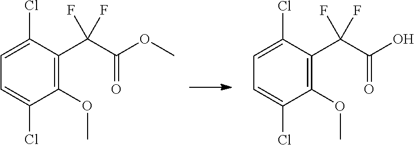 [SMILES: COC(=O)C(F)(F)c1c(Cl)ccc(Cl)c1OC ; COc1c(Cl)ccc(Cl)c1C(F)(F)C(=O)O]

To the compound methyl 2-(3,6-dichloro-2-methoxy-phenyl)-2,2-difluoro-acetate (1.50 g, 5.26 mmol) in dry tetrahydrofuran (15 mL) and water (15 mL) was added lithium hydroxide (0.662 g, 15.8 mmol). The reaction mixture was stirred at room temperature for 1 hour. The reaction mixture was extracted with ethyl acetate. The aqueous layer was acidified with 2 N hydrochloric acid and extracted with ethyl acetate. The combined organic layers were dried over sodium sulfate and evaporated under reduced pressure. The crude material was triturated with n-pentane to give 2-(3,6-dichloro-2-methoxy-phenyl)-2,2-difluoro-acetic acid (0.778 g, 2.87 mmol, 55%).

¹H NMR (400 MHz, DMSO-d6): δ 15.4-14.8 (br s, 1H), 7.75-7.73 (d, 1H), 7.44-7.42 (d, 1H), 3.79 (s, 3H) ppm

Also prepared by this general method were:

2-(2-Chloro-6-methoxy-phenyl)-2,2-difluoro-acetic acid (Compound 1-65)

¹H NMR (400 MHz, DMSO-d6): δ 14.38 (br s, 1H), 7.52-7.48 (t, 1H), 7.19-7.15 (t, 2H), 3.78 (s, 3H) ppm

### Example 4 Preparation of 2-(5-acetamido-2, 3-dichlorophenyl)-2, 2-difluoroacetic acid (Compound 1-77)

### Step 1 Synthesis of N-(3-bromo-4,5-dichlorophenyl)acetamide

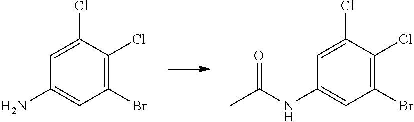

3-bromo-4,5-dichloroaniline (0.70 g, 2.90 mmol) and triethylamine (0.63 mL, 4.35 mmol) in CH2Cl2 (10.0 mL) was charged with AcCl (0.31 g, 4.35 mmol) at 0° C. The reaction mixture was stirred at room temperature for 2 h. Upon completion, the reaction mixture was diluted with CH2Cl2 (50 mL) and washed with H2O (20 mL) followed by brine solution (20 mL), organic layer dried over anhydrous Na₂SO₄, filtered and concentrated under reduced pressure to obtain crude product. The obtained crude product was purified by column chromatography (silica gel using 30-50% EtOAc in hexanes as eluent) to afford N-(3-bromo-4,5-dichlorophenyl) acetamide (0.70 g, 85% yield, AMRI lot #IN-KUC-A-28-1) as an off white solid.

### Step 2 Synthesis of ethyl 2-(5-acetamido-2,3-dichlorophenyl)-2,2-difluoroacetate (Compound 3-77)

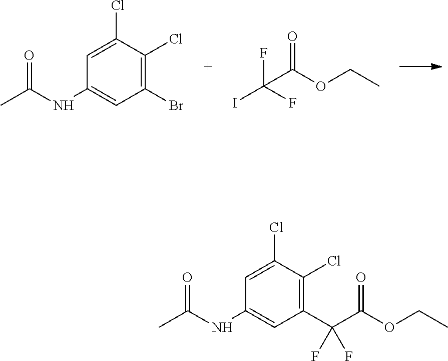

N-(3-bromo-4,5-dichlorophenyl)-acetamide (0.61 g, 2.15 mmol) and ethyl 2,2-difluoro-2-iodoacetate (0.80 g, 3.23 mmol) in DMSO (6.0 mL) was added Cu powder (1.1 g, 17.24 mmol) at room temperature. The reaction mixture was stirred at 60° C. for 5 h in microwave. The progress of the reaction was monitored by thin-layer chromatography (TLC). Upon completion, the reaction mixture was filtered through a pad of celite and washed with EtOAc (50.0 mL). Filtrate was washed with ice cold H2O (2×20.0 mL) followed by brine solution (40.0 mL), organic layer dried over anhydrous Na2SO4, filtered and concentrated under reduced pressure to obtain crude product. The obtained crude product was purified by column chromatography (silica gel using 15-30% EtOAc in hexanes as eluent) to afford ethyl 2-(5-acetamido-2, 3-dichlorophenyl)-2, 2-difluoroacetate (0.31 g, 42% yield) as a clear liquid.

### Step 3 Synthesis of 3-[carboxy(difluoro)methyl]-4,5-dichloroanilinium (Compound 1-77)

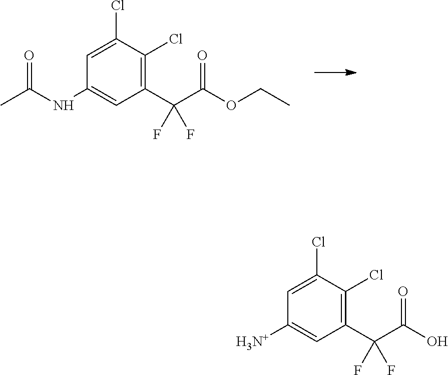 [SMILES: CCOC(=O)C(F)(F)c1cc(NC(C)=O)cc(Cl)c1Cl ; [NH3+]c1cc(Cl)c(Cl)c(C(F)(F)C(=O)O)c1]

Ethyl 2-(5-acetamido-2, 3-dichlorophenyl)-2, 2-difluoroacetate (0.28 g, 0.85 mmol), in conc. HCl:H2O (3:1, 13 mL) was stirred at 100° C. for 16 h. Upon completion, excess of H2O was concentrated under reduced pressure to afford 3-[carboxy(difluoro)methyl]-4,5-dichloroanilinium (HCl salt) (100 mg, 40% yield) as an off white solid.

1H NMR (400 MHz, DMSO-d6): δ 6.93-6.89 (m, 5H) ppm

Also prepared by this general method were:

2-(5-Amino-2-chloro-phenyl)-2,2-difluoro-acetic acid (Compound 1-63)

¹H NMR (400 MHz, DMSO-d6): δ 7.13 (d, 1H), 6.91 (s, 1H), 6.66 (dd, 1H) ppm

Ammonium;2-(4-amino-2,3-dichloro-phenyl)-2,2-difluoro-acetate (Compound 7-75)

¹H NMR (400 MHz, DMSO-d6): δ 7.3 (br s, 4H), 7.2 (d, 1H), 6.7 (d, 1H), 5.84 (br s, 2H) ppm

### Example 5 Preparation of 2-(3,5-dimethylbenzo[d]isoxazol-4-yl)-2,2-difluoroacetic acid (Compound 1-396)

### Step 1 Synthesis of 2-(1-iminoethyl)-4-methylphenol

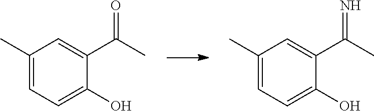

1-(2-Hydroxy-5-methylphenyl)ethanone (1.00 g, 6.66 mmol) in MeOH (5.0 mL) was charged with 7 M ammonia in MeOH (10.0 mL). The reaction mixture was sealed and stirred at room temperature for 4 h. Upon completion of reaction, yellow solid formation was observed. The reaction mass filtered off to provide 2-(1-iminoethyl)-4-methylphenol (0.70 g, crude) as a yellow solid.

1H NMR (400 MHz, CDCl3): δ 14.8 (brs, 1H), 9.15 (brs, 1H), 7.27 (d, 1H), 7.14 (dd, 1H), 6.87 (d, 1H), 2.46 (s, 3H), 2.28 (s, 3H) ppm

### Step 2 Synthesis of 3,5-dimethylbenzo[d]isoxazole

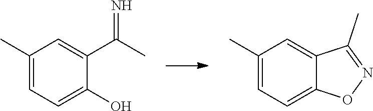

2-(1-Iminoethyl)-4-methylphenol (0.65 g, crude) in THE (10.0 mL) was charged with K2CO3 (0.93 g, 6.72 mmol) and N-chlorosuccinamide (0.70 g, 5.40 mmol). The reaction mixture was stirred at room temperature for 18 h. Upon completion of reaction, the reaction mass was diluted with H2O (20 mL) and extracted with EtOAc (2×20 mL). The combined layers were washed with brine (10 mL), dried over anhydrous Na2SO4 and concentrated under reduced pressure to obtain residue. The resultant residue purified by flash chromatography (20% EtOAc/hexanes eluent) to afford 3,5-dimethylbenzo[d]isoxazole (600 mg, 4.08 mmol) as an off-white solid.

1H NMR (400 MHz, CDCl3): δ 7.44-7.33 (m, 3H), 2.55 (s, 3H), 2.47 (s, 3H) ppm

### Step 3 Synthesis of ethyl 2-(3,5-dimethylbenzo[d]isoxazol-4-yl)-2,2-difluoroacetate (Compound 3-396)

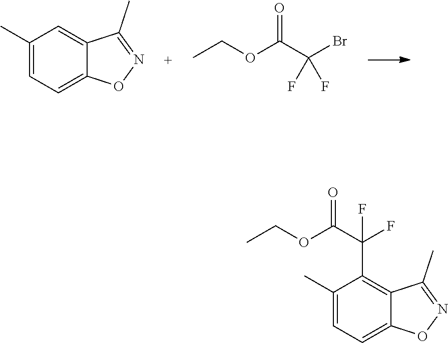

3,5-Dimethylbenzo[d]isoxazole (600 mg, 4.08 mmol) in DMSO (20 mL) was charged with ethyl 2,2-difluoro-2-bromoacetate (1.66 g, 8.16 mmol), K3PO4 (1.30 g, 6.12 mmol) and fac-[Ir(PPy3)] (80 mg, 0.12 mmol). The reaction mixture was sealed and stirred at room temperature for 48 h under the exposure of 450-455 nm blue light. Upon completion of reaction, the reaction mass was diluted with H2O (20 mL) and filtered through celite pad. The filtrate was extracted with tertiary butyl methyl ether (2×10 mL). The combined layers were washed with brine (10 mL), dried over anhydrous Na2SO4 and concentrated under reduced pressure to obtain crude material. The resultant residue was purified by flash chromatography (10-15% EtOAc/hexanes eluent) to provide ethyl 2-(3,5-dimethylbenzo[d]isoxazol-4-yl)-2,2-difluoroacetate (300 mg, 27%) as an off-white solid.

1H NMR (400 MHz, CDCl3): δ 7.57 (d, 1H), 7.44-7.33 (m, 1H), 4.32 (q, 2H), 2.67 (t, 3H), 2.58 (t, 3H), 1.30 (t, 3H) ppm

### Step 4 Synthesis of 2-(3,5-dimethylbenzo[d]isoxazol-4-yl)-2,2-difluoroacetic acid (Compound 1-396)

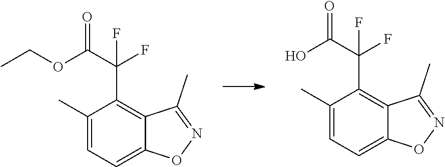 [SMILES: CCOC(=O)C(F)(F)c1c(C)ccc2onc(C)c12 ; Cc1ccc2onc(C)c2c1C(F)(F)C(=O)O]

Ethyl 2-(3,5-dimethylbenzo[d]isoxazol-4-yl)-2,2-difluoroacetate (300 mg, 1.67 mmol) in THF:H2O (3:1, 12 mL) was charged with LiOH·H2O (140 mg, 3.34 mmol). The reaction mixture was stirred at room temperature for 2 h. Upon completion of reaction, the reaction mass evaporated and diluted with H2O (5 mL). Then, the aqueous layer was acidified to pH=3 using aqueous 2 N HCl and extracted into MTBE (2×15 mL). The combined layers were dried over anhydrous Na2SO4 and concentrated under reduced pressure to afford 2-(3,5-dimethylbenzo[d]isoxazol-4-yl)-2,2-difluoroacetic acid (300 mg, 75%) as an off-white solid.

1H NMR (400 MHz, DMSO-d6): δ 7.83 (d, 1H), 7.60 (d, 1H), 2.59 (t, 3H), 2.54 (t, 3H) ppm

Also prepared by this general method were:

2,2-Difluoro-2-(3-methyl-1,2-benzoxazol-4-yl)acetic acid (Compound 1-391)

¹H NMR (400 MHz, DMSO-d6): δ 7.99 (d, 1H), 7.79 (t, 1H), 7.59 (d, 1H), 2.60 (t, 3H) ppm

2-(5-Chloro-3-methyl-1,2-benzoxazol-4-yl)-2,2-difluoro-acetic acid (Compound 1-395)

¹H NMR (400 MHz, DMSO-d6): δ 8.03 (d, 1H), 7.86 (d, 1H), 2.57 (t, 3H) ppm

### Example 6 Preparation of 2-(2-chloronaphthalen-1-yl)-2,2-difluoroacetic acid (Compound 1-401)

### Step 1 Synthesis of ethyl 2-(2-chloronaphthalen-1-yl)-2,2-difluoroacetate (Compound 3-401)

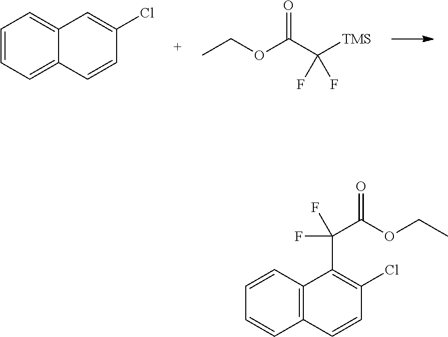 [SMILES: Clc1ccc2ccccc2c1 ; CCOC(=O)C(F)(F)[Si](C)(C)C ; CCOC(=O)C(F)(F)c1c(Cl)ccc2ccccc12]

2-Chloronaphthalene (1.00 g, 6.17 mmol) in 1,2-dichloroethane (20 mL) was charged with ethyl 2,2-difluoro-2-(trimethylsilyl)acetate (3.00 g, 15.4 mmol), AgOTf (6.34 g, 24.7 mmol) and KF (1.43 g, 24.7 mmol). The reaction mixture was sealed and exposed to microwave irradiation at 60° C. for 1 h. Upon completion of reaction, the reaction mass evaporated. The residue was diluted with H2O (50 mL) and extracted into EtOAc (2×50 mL). The combined layers were washed with brine (10 mL), dried over anhydrous Na2SO4 and concentrated under reduced pressure to obtain crude material. The obtained crude material was purified by flash chromatography (10-20% EtOAc/hexanes as eluent) to provide ethyl 2-(2-chloronaphthalen-1-yl)-2,2-difluoroacetate (120 mg) as an off-white solid which was used directly in the next step.

Also prepared by this general method was:

Ethyl 2,2-difluoro-2-(1-naphthyl)acetate (Compound 3-397)

¹H NMR (400 MHz, DMSO-d6): δ 8.18 (d, 1H), 8.08 (dd, 1H), 8.02 (dd, 1H), 7.87 (d, 1H), 7.70-7.62 (m, 3H), 4.32 (q, 2H), 1.15 (t, 3H) ppm

### Step 2 Synthesis of 2-(2-chloronaphthalen-1-yl)-2,2-difluoroacetic acid (Compound 1-401)

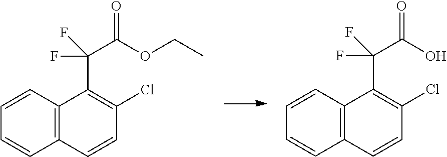 [SMILES: CCOC(=O)C(F)(F)c1c(Cl)ccc2ccccc12 ; O=C(O)C(F)(F)c1c(Cl)ccc2ccccc12]

Ethyl 2-(2-chloronaphthalen-1-yl)-2,2-difluoroacetate (150 mg, 5.28 mmol) in THF:H2O (3:1, 12 ml) was charged with LiOH·H2O (44 mg, 10.6 mmol). The reaction mixture was stirred at room temperature for 3 h. Then, reaction mass evaporated under reduced pressure and diluted with H2O (5 mL). Then, the aqueous layer acidified to pH=5 using aqueous 1 N HCl and evaporated under reduced pressure to obtain crude material. The crude was purified by reverse phase column chromatography (50% CH3CN H2O as eluent) to afford 2-(2-chloronaphthalen-1-yl)-2,2-difluoroacetic acid (15 mg, 1.1%) as an off-white solid.

1H NMR (400 MHz, DMSO-d6): δ 8.42 (d, 1H), 7.94-7.87 (m, 2H), 7.53-7.37 (m, 2H), 7.45 (d, 1H) ppm

### Example 7 Preparation of 2-(2-amino-3,6-dichlorophenyl)-2,2-difluoroacetic acid (Compound 7-254)

### Step 1 Synthesis of ethyl 2-(2-amino-3,6-dichlorophenyl)-2,2-difluoro acetate and 4,7-dichloro-3,3-difluoroindolin-2-one

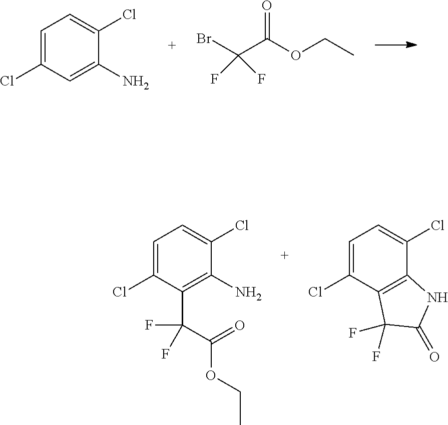

To a solution of 2,5-dichloroaniline (300 mg, 2.2 mmol) in DMSO (5 mL) was added ethyl 2-bromo-2,2-difluoroacetate (447 mg, 6.6 mmol) followed by addition of ferrocene (41 mg, 0.22 mmol). The reaction mixture was cooled to 0° C. and a solution of 30% H2O2 (0.45 mL, 4.4 mmol) was added dropwise. The reaction mixture was slowly warmed to ambient temperature and continued stirring at same temperature overnight. Upon completion of reaction, reaction mixture was quenched with H2O (5 mL), extracted with EtOAc (2×10 mL). The organic layer was washed with brine (10 mL) and dried over Na₂SO₄ and evaporated under reduced pressure to obtain crude material. The obtained crude material was purified by combi flash chromatography (0-15% EtOAc:hexane) to afford a mixture of 1 & 2 (1:1 ratio by UPLC-MS) (0.20 g) as yellow solid.

1H NMR (400 MHz, CDCl3): δ 8.0 (brs, 1H), 7.6 (s, 1H), 7.38 (dt, 1H), 7.07 (d, 1H), 6.7 (s, 1H), 4.34 (q, 2H), 1.32 (t, 3H) ppm

### Step 2 Synthesis of 2-(2-amino-3,6-dichlorophenyl)-2,2-difluoroacetic acid (Compound 7-254)

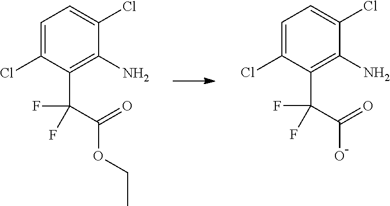 [SMILES: CCOC(=O)C(F)(F)c1c(Cl)ccc(Cl)c1N ; Nc1c(Cl)ccc(Cl)c1C(F)(F)C(=O)O]

Starting materials containing a mixture of ethyl 2-(2-amino-3,6-dichlorophenyl)-2,2-difluoro acetate and 4,7-dichloro-3,3-difluoroindolin-2-one (200 mg, 0.834 mmol) were taken in a 3-necked round bottom flask with THF:H2O (5.0 mL: 2.5 mL). KOH pellets (237 mg, 4.21 mmol) were added to the reaction mixture. This reaction mixture was heated at 55° C. for overnight. Upon completion of reaction, solvents were evaporated and submitted for prep-HPLC purification without any acid-base work up since in presence of acid, product again getting transformed into its cyclized form. In prep-HPLC, product was purified using ammonium bicarbonate buffer and thus it was isolated in ammonium salt form (0.10 g, 47%) as pale yellow solid.

¹H NMR (400 MHz, DMSO-d6): δ 7.2 (d, 1H), 7.15 (brs, 4H), 6.6 (d, 1H), 6.12 (brs, 2H) ppm

Also prepared by this general method were:

2-(3,6-Difluoro-2-methoxyphenyl)-2,2-difluoroacetic acid (Compound 1-314)

¹H-NMR (400 MHz, DMSO-d6): δ 7.61-7.47 (m, 1H), 7.37-7.29 (m, 1H), 3.90 (s, 3H) ppm

2,2-Difluoro-2-(3-fluoro-2-methoxy-phenyl)acetic acid (Compound 1-306)

¹H-NMR (400 MHz, DMSO-d6): δ 7.41-7.36 (m, 2H), 7.33-7.29 (m, 1H), 3.89 (s, 3H) ppm

2-(2-Amino-3-chloro-phenyl)-2,2-difluoro-acetic acid (Compound 1-252)

¹H NMR (400 MHz, DMSO-d6): δ 7.24 (d, 1H), 7.17 (d, 1H), 6.56-6.52 (m, 1H), 6.10 (br s, 2H) ppm

2-(2-Amino-3,5,6-trichloro-phenyl)-2,2-difluoro-acetic acid (Compound 1-81)

¹H NMR (400 MHz, DMSO-d6): δ 7.58 (s, 1H), 6.18 (s, 2H) ppm

Ammonium 2-(2-amino-3,5-dichloro-phenyl)-2,2-difluoro-acetate (Compound 7-253)

¹H NMR (400 MHz, DMSO-d6): δ 7.38 (s, 1H), 7.13 (m, 1H), 7.10 (s, 4H), 6.24 (s, 2H) ppm

Lithium;2-(2-amino-5-chloro-phenyl)-2,2-difluoro-acetate (Compound 5-251)

¹H NMR (400 MHz, DMSO-d6): δ 7.08-7.07 (m, 1H), 7.06-7.03 (m, 1H), 6.62-6.60 (m, 1H), 5.95 (s, 2H) ppm

Lithium;2-(6-amino-2,3-dichloro-phenyl)-2,2-difluoro-acetate (Compound 5-80)

¹H NMR (400 MHz, DMSO-d6): δ 7.19 (d, 1H), 6.61 (d, 1H), 5.89 (s, 2H) ppm

Ethyl 2-(3,6-difluoro-2-hydroxyphenyl)-2,2-difluoroacetate (Compound 3-297)

¹H NMR (400 MHz, DMSO-d6): δ 11.20 (s, 1H), 7.57-7.47 (m, 1H), 6.94-6.88 (m, 1H), 4.33 (q, 2H), 1.22 (t, 3H) ppm

2-(4,5-Dichloro-2-hydroxy-phenyl)-2,2-difluoro-acetic acid (Compound 1-298)

¹H NMR (400 MHz, DMSO-d6): δ 7.64 (s, 1H), 7.27 (s, 1H) ppm

2-(3-Chloro-6-fluoro-2-methoxyphenyl)-2,2-difluoroacetic acid (Compound 7-325)

¹H NMR (400 MHz, DMSO-d6): δ 7.55 (s, 1H), 7.26 (bs, 4H), 7.07-7.02 (m, 1H), 3.75 (s, 3H) ppm

Ammonium;2-(5-chloro-2-fluoro-4-methoxy-phenyl)-2,2-difluoro-acetate (Compound 7-31)

¹H NMR (400 MHz, DMSO-d6): δ 7.43-7.41 (m, 1H), 7.14-7.05 (m, 5H), 3.87 (s, 3H) ppm

Compounds 1-3, 1-9, 1-32, 1-263, 5-255, 1-7, 1-341, 1-304, 1-20, 1-328, 1-1, 1-305, 1-186, 1-6, 1-2 and 1-473 are obtainable as of the priority date of the present application from commercial sources, such as Enamine, Accela and ChemDiv.

### Formulation Examples

| Wettable powders | a) | b) | c) |
|---|---|---|---|
| active ingredients | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The combination is thoroughly mixed with the adjuvants and the mixture is thoroughly ground in a suitable mill, affording wettable powders that can be diluted with water to give suspensions of the desired concentration.

Emulsifiable concentrate

| | |
|---|---|
| active ingredients | 10% |
| octylphenol polyethylene glycol ether | 3% |
| (4-5 mol of ethylene oxide) | |
| calcium dodecylbenzenesulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredients | 5% | 6% | 4% |
| Talcum | 95% | — | — |
| Kaolin | — | 94% | — |
| mineral filler | — | — | 96% |

Ready-for-use dusts are obtained by mixing the combination with the carrier and grinding the mixture in a suitable mill.

Extruder granules

| | |
|---|---|
| Active ingredients | 15% |
| sodium lignosulfonate | 2% |
| carboxymethylcellulose | 1% |
| Kaolin | 82% |

The combination is mixed and ground with the adjuvants, and the mixture is moistened with water. The mixture is extruded and then dried in a stream of air.

Coated granules

| | |
|---|---|
| Active ingredients | 8% |
| polyethylene glycol (mol. wt. 200) | 3% |
| Kaolin | 89% |

The finely ground combination is uniformly applied, in a mixer, to the kaolin moistened with polyethylene glycol. Non-dusty coated granules are obtained in this manner.

Suspension concentrate

| | |
|---|---|
| active ingredients | 40% |
| propylene glycol | 10% |
| nonylphenol polyethylene glycol ether (15 mol of ethylene oxide) | 6% |
| Sodium lignosulfonate | 10% |
| carboxymethylcellulose | 1% |
| silicone oil (in the form of a 75% emulsion in water) | 1% |
| Water | 32% |

The finely ground combination is intimately mixed with the adjuvants, giving a suspension concentrate from which suspensions of any desired dilution can be obtained by dilution with water.

### Slow Release Capsule Suspension

28 parts of the combination are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed.

The obtained capsule suspension is stabilized by adding 0.25 parts of a thickener and 3 parts of a dispersing agent. The capsule suspension formulation contains 28% of the active ingredients. The medium capsule diameter is 8-15 microns.

The resulting formulation is applied to seeds as an aqueous suspension in an apparatus suitable for that purpose.

### Biological Examples

### Biological Efficacy

Seeds of a variety of test species were shown in unsterilised compost in small pots. After cultivation for one day (pre-emergence) or seven days (post-emergence) in controlled conditions in the glasshouse (at 24/16° C., day/night; 14 hours light; 65% humidity) the plants were sprayed with 1 mg of the active ingredient, formulated in 466 μl of a acetone/water/Tween 20 (49.75:49.75:0.5) solution, which is equivalent to 1000 g/ha. Once the foliage was dry, the pots were kept in the glasshouse (at 24/16° C., day/night; 14 hours light; 65% humidity), and were watered twice daily. After 12 days the test was evaluated and scored (100=total damage to plant, 0=no damage to plant). The results are shown in Table 2 below.

TABLE 2

| | | Species | | | | | | | |
|---|---|---|---|---|---|---|---|---|---|
| | Rate | Post-Emergence | | | | Pre-Emergence | | | |
| Compound | (g/ha) | AMARE | LOLPE | STEME | DIGSA | AMARE | LOLPE | STEME | DIGSA |
| 1-1 | 1000 | 40 | 0 | 0 | 0 | 0 | 0 | 20 | 0 |
| 1-2 | 1000 | 40 | 0 | 50 | 0 | 0 | 0 | 0 | 0 |
| 1-3 | 1000 | 70 | 0 | 30 | 0 | 50 | 30 | 30 | 0 |
| 1-6 | 1000 | 80 | 0 | 20 | 0 | — | 0 | 0 | 0 |
| 1-7 | 1000 | 40 | 0 | 70 | 0 | 0 | 0 | 30 | 0 |
| 1-8 | 1000 | 10 | 0 | 10 | 0 | 0 | 0 | 0 | 0 |
| 1-9 | 1000 | 90 | 0 | 80 | 0 | 80 | 0 | 60 | 0 |
| 1-20 | 1000 | 70 | 0 | 0 | 30 | 30 | 0 | 0 | 0 |
| 1-32 | 1000 | 30 | 0 | 40 | 0 | 0 | 0 | 0 | 0 |
| 1-186 | 1000 | 60 | 0 | 30 | 0 | 10 | 0 | 10 | 0 |
| 1-263 | 1000 | 50 | 0 | 50 | 0 | 40 | 0 | 0 | 0 |
| 1-275 | 1000 | 90 | 0 | 80 | 30 | 100 | 60 | 100 | 80 |
| 1-291 | 1000 | 100 | 0 | 80 | 70 | 90 | 80 | 100 | 90 |
| 1-304 | 1000 | 70 | 20 | 20 | 20 | 0 | 0 | 0 | 0 |
| 1-305 | 1000 | 50 | 0 | 0 | 0 | 0 | 0 | 0 | 0 |
| 1-328 | 1000 | 80 | 0 | 60 | 0 | 50 | 0 | 40 | 0 |
| 1-341 | 1000 | 80 | 10 | 60 | 20 | 30 | 10 | 30 | 0 |
| 1-397 | 1000 | 70 | 0 | 80 | 0 | 90 | 70 | 90 | 30 |
| 3-397 | 1000 | 40 | 0 | 70 | 0 | 0 | 0 | — | 0 |
| 3-419 | 1000 | 80 | 0 | 70 | 40 | 80 | 0 | 60 | 0 |
| 5-255 | 1000 | 70 | 0 | 70 | 0 | 20 | 0 | — | 0 |
| 7-31 | 1000 | 100 | 0 | 0 | 0 | 0 | 0 | 0 | 0 |
| 7-231 | 1000 | 90 | 0 | 80 | 70 | 90 | 40 | 100 | 80 |

### Pre-Emergence Biological Efficacy

Seeds of weeds and/or crops were sown in standard soil in pots. After cultivation for one day under controlled conditions in a glasshouse (at 24/19° C., day/night; 16 hours light), the plants were sprayed with an aqueous spray solution derived from the formulation of the technical active ingredient in a small amount of acetone and a special solvent and emulsifier mixture referred to as AMA (11.12% Emulsogen EL360 TM+44.44% N-methylpyrrolidone+44.44% Dowanol DPM glycol ether), to create a 50 g/l solution which was then diluted using 0.2% Genapol X080 as diluent to give the desired final dose of test compound.

The test plants were then grown under controlled conditions in the glasshouse (at 24/18° C., day/night; 15 hours light; 50% humidity) and watered twice daily. After 13 days the test was evaluated (100=total damage to plant; 0=no damage to plant). The results are shown in Table 3 below.

TABLE 3

| | Rate | Species | | | | | | | | | |
|---|---|---|---|---|---|---|---|---|---|---|---|
| Compound | (g/ha) | AMARE | AMAPA | STEME | ABUTH | ECHCG | ZEAMX | IPOHE | SOLNI | SETFA | LOLPE |
| 1-5 | 1000 | 10 | — | 20 | 10 | — | 10 | 20 | — | — | — |
| 1-9 | 1000 | 70 | — | — | — | 20 | — | 60 | 90 | 20 | 20 |
| 1-59 | 250 | 20 | 20 | — | — | 0 | 0 | 10 | — | 10 | — |
| 1-61 | 1000 | 20 | — | 0 | 0 | — | 0 | 0 | — | — | — |
| 1-62 | 1000 | 80 | — | 80 | 50 | 30 | 20 | 20 | — | — | — |
| 1-63 | 1000 | 0 | — | 0 | 0 | 0 | 0 | 0 | — | — | — |
| 1-64 | 1000 | 0 | — | 0 | 0 | — | 10 | 10 | — | — | — |
| 1-65 | 1000 | 60 | — | — | — | 0 | — | 40 | — | 0 | 0 |
| 1-67 | 1000 | 0 | — | 0 | 0 | — | 10 | 30 | — | — | — |
| 1-77 | 1000 | 10 | — | 10 | 10 | — | 0 | 30 | — | — | — |
| 1-78 | 1000 | 0 | — | 0 | — | — | 0 | 0 | — | — | — |
| 1-81 | 1000 | 30 | — | 20 | 20 | 0 | 0 | 10 | — | — | — |
| 1-82 | 1000 | 60 | — | 30 | — | — | 0 | 20 | — | — | — |
| 1-190 | 1000 | 80 | 80 | — | — | 0 | 0 | 90 | — | 20 | — |
| 1-192 | 1000 | 80 | 70 | — | — | 0 | 0 | 70 | — | 50 | — |
| 1-196 | 1000 | 40 | — | 80 | 70 | — | 20 | 70 | — | — | — |
| 1-207 | 1000 | 80 | — | 90 | 90 | — | 30 | 90 | — | — | — |
| 1-208 | 1000 | 90 | — | 100 | 100 | — | 80 | 90 | — | — | — |
| 1-209 | 1000 | 70 | — | 60 | 60 | 0 | 0 | 80 | — | — | — |
| 1-220 | 1000 | 50 | — | 50 | 80 | 0 | 10 | 70 | — | — | — |
| 1-235 | 1000 | 40 | — | 30 | 30 | 10 | 0 | 20 | — | — | — |
| 1-236 | 1000 | 20 | — | 0 | 0 | 0 | — | 0 | — | — | — |
| 1-238 | 1000 | 0 | — | 0 | 0 | 0 | 0 | 0 | — | — | — |
| 1-242 | 1000 | 70 | 70 | — | — | 0 | 10 | 60 | — | 10 | — |
| 1-244 | 1000 | 90 | 80 | — | — | 50 | 40 | 90 | — | 90 | — |
| 1-252 | 1000 | 40 | — | 10 | 90 | 0 | 0 | 50 | — | — | — |
| 1-261 | 1000 | 40 | 50 | — | — | 10 | 0 | 40 | — | 10 | — |
| 1-262 | 1000 | 90 | — | 100 | 90 | 0 | 40 | 90 | — | — | — |
| 1-298 | 1000 | 0 | — | 0 | 0 | — | 0 | 0 | — | — | — |
| 1-306 | 1000 | 30 | — | 10 | 20 | 10 | 0 | 60 | — | — | — |
| 1-314 | 1000 | 0 | — | 0 | 0 | 0 | 0 | 0 | — | — | — |
| 1-317 | 1000 | 90 | — | 100 | 100 | 90 | 70 | 90 | — | — | — |
| 1-326 | 1000 | 50 | — | — | — | 30 | — | 20 | — | 10 | 10 |
| 1-356 | 1000 | 50 | — | 30 | 30 | 0 | 10 | 50 | — | — | — |
| 1-359 | 1000 | 10 | — | 0 | 0 | — | 10 | 0 | — | — | — |
| 1-385 | 1000 | 60 | — | 50 | 90 | 50 | 50 | 70 | — | — | — |
| 1-389 | 1000 | 60 | — | 60 | 50 | 10 | 10 | 60 | — | — | — |
| 1-391 | 1000 | 70 | — | 20 | 40 | 40 | 10 | 60 | — | — | — |
| 1-395 | 1000 | 30 | — | 10 | 10 | 20 | 0 | 50 | — | — | — |
| 1-396 | 1000 | 0 | — | 0 | 0 | 0 | 30 | 0 | — | — | — |
| 1-397 | 1000 | 80 | — | — | — | 60 | — | 80 | 100 | 20 | 40 |
| 1-401 | 1000 | 80 | — | 80 | 60 | 30 | 0 | 50 | — | — | — |
| 1-403 | 1000 | 80 | — | 70 | 80 | — | 30 | 90 | — | — | — |
| 1-406 | 1000 | 80 | — | 50 | 60 | 0 | 10 | 60 | — | — | — |
| 1-407 | 1000 | 60 | — | 10 | 70 | 50 | 40 | 60 | — | — | — |
| 1-409 | 1000 | 50 | — | 30 | 70 | 20 | 0 | 70 | — | — | — |
| 1-416 | 1000 | 90 | — | 80 | 80 | 40 | 10 | 90 | — | — | — |
| 1-418 | 1000 | 30 | — | — | — | 20 | — | 50 | 10 | 50 | 0 |
| 1-420 | 1000 | 80 | 60 | — | — | 0 | 10 | 70 | — | 0 | — |
| 1-436 | 1000 | 30 | 40 | — | — | 0 | 0 | 40 | — | 20 | — |
| 1-428 | 1000 | 80 | 90 | — | — | 0 | 0 | 80 | — | 0 | — |
| 1-444 | 1000 | 40 | 60 | — | — | 0 | 0 | 50 | — | 0 | — |
| 1-452 | 1000 | 50 | 50 | — | — | 10 | 30 | 70 | — | 30 | — |
| 1-458 | 1000 | 90 | 90 | — | — | 0 | 20 | 80 | — | 0 | — |
| 1-459 | 1000 | 70 | 80 | — | — | 50 | 0 | 90 | — | 100 | — |
| 1-460 | 1000 | 80 | 90 | — | — | 0 | 10 | 70 | — | 10 | — |
| 1-461 | 1000 | 40 | 30 | — | — | 0 | 10 | 20 | — | 30 | — |
| 1-462 | 1000 | 50 | 30 | — | — | 0 | 0 | 40 | — | 0 | — |
| 1-463 | 1000 | 50 | 60 | — | — | 0 | 0 | 50 | — | 0 | — |
| 1-464 | 250 | 40 | 60 | — | — | 10 | 20 | 50 | — | 30 | — |
| 1-465 | 1000 | 70 | 70 | | | 20 | 20 | 20 | | 20 | |
| 2-65 | 1000 | 50 | — | — | — | 0 | — | 70 | 10 | 0 | 0 |
| 2-326 | 1000 | 40 | — | — | — | 20 | — | 40 | — | 10 | 10 |
| 2-418 | 1000 | 60 | — | — | — | 10 | — | 50 | — | 20 | 0 |
| 3-67 | 1000 | 10 | — | 10 | 0 | — | 0 | 20 | — | — | — |
| 3-297 | 1000 | 0 | — | 0 | 0 | — | 0 | 0 | — | — | — |
| 3-397 | 1000 | 10 | — | 40 | 50 | 0 | 20 | 0 | — | — | — |
| 3-419 | 1000 | 20 | — | 40 | 30 | 0 | 0 | 10 | — | — | — |
| 5-80 | 1000 | 0 | — | 0 | 0 | 0 | 0 | 0 | — | — | — |
| 5-251 | 1000 | 20 | — | 10 | 10 | — | 0 | 0 | — | — | — |
| 7-31 | 1000 | 0 | — | 0 | 0 | — | 0 | 0 | — | — | — |
| 7-75 | 1000 | 70 | — | 70 | 70 | 30 | 0 | 20 | — | — | — |
| 7-231 | 1000 | 90 | — | 100 | 90 | 10 | 50 | 80 | — | — | — |
| 7-237 | 1000 | 0 | — | 0 | 10 | 0 | 0 | 50 | — | — | — |
| 7-250 | 1000 | 100 | — | 100 | 90 | 30 | 50 | 90 | — | — | — |
| 7-253 | 1000 | 0 | — | — | 0 | 0 | — | 0 | — | — | — |
| 7-254 | 1000 | 20 | — | 30 | 40 | 0 | 10 | 10 | — | — | — |
| 7-325 | 1000 | 50 | — | 30 | 20 | — | 10 | 50 | — | — | — |

### Post-Emergence Biological Efficacy

Seeds of weeds and/or crops were sown in standard soil in pots. After cultivation for 14 days under controlled conditions in a glasshouse (at 24/19° C., day/night; 16 hours light), the plants were sprayed with an aqueous spray solution derived from the formulation of the technical active ingredient in a small amount of acetone and a special solvent and emulsifier mixture referred to as F50 (11.12% Emulsogen EL360 TM+44.44% N-methylpyrrolidone+44.44% Dowanol DPM glycol ether), to create a 50 g/l solution which was then diluted using 0.2% Genapol X080 as diluent to give the desired final dose of test compound.

The test plants were then grown under controlled conditions in the glasshouse (at 24/18° C., day/night; 15 hours light; 50% humidity) and watered twice daily. After 13 days the test was evaluated (100=total damage to plant; 0=no damage to plant). The results are shown in Table 4 below.

TABLE 4

| | Rate | Species | | | | | | | | | |
|---|---|---|---|---|---|---|---|---|---|---|---|
| Compound | (g/ha) | AMARE | AMAPA | STEME | ABUTH | ECHCG | ZEAMX | IPOHE | SOLNI | SETFA | LOLPE |
| 1-5 | 1000 | 20 | — | 20 | — | 10 | 10 | 20 | — | — | — |
| 1-9 | 1000 | 70 | — | — | — | 0 | — | 60 | 60 | 0 | 0 |
| 1-59 | 250 | 60 | 60 | — | — | 0 | 0 | 40 | — | 10 | — |
| 1-61 | 1000 | 10 | — | 10 | 10 | — | 10 | 20 | — | — | — |
| 1-62 | 1000 | 60 | — | 60 | 20 | — | 20 | 70 | — | — | — |
| 1-63 | 1000 | 20 | — | 0 | 10 | 10 | 0 | 20 | — | — | — |
| 1-64 | 1000 | 50 | — | 0 | 0 | — | 40 | 50 | — | — | — |
| 1-65 | 1000 | 70 | — | — | — | 0 | — | 70 | — | 0 | 0 |
| 1-67 | 1000 | 30 | — | 10 | 10 | — | 10 | 60 | — | — | — |
| 1-77 | 1000 | 60 | — | 30 | — | 0 | 10 | 10 | — | — | — |
| 1-78 | 1000 | 50 | — | 0 | 10 | — | 30 | 40 | — | — | — |
| 1-81 | 1000 | 50 | — | 50 | 20 | 10 | 0 | 50 | — | — | — |
| 1-82 | 1000 | 60 | — | 10 | 20 | — | 20 | 30 | — | — | — |
| 1-190 | 1000 | 90 | 80 | — | — | 40 | 10 | 60 | — | 20 | — |
| 1-192 | 1000 | 80 | 80 | — | — | 30 | 20 | 70 | — | 60 | — |
| 1-196 | 1000 | 50 | — | 90 | 80 | — | 10 | 70 | — | — | — |
| 1-207 | 1000 | 70 | — | 90 | 80 | — | 30 | 80 | — | — | — |
| 1-208 | 1000 | 90 | — | 90 | 90 | | 70 | 90 | — | — | — |
| 1-209 | 1000 | 80 | — | 90 | — | 10 | 10 | 80 | — | — | — |
| 1-220 | 1000 | 70 | — | 70 | — | 0 | 10 | 70 | — | — | — |
| 1-235 | 1000 | 70 | — | 50 | — | 20 | 20 | 30 | — | — | — |
| 1-236 | 1000 | 30 | — | 10 | 0 | 0 | 10 | 30 | — | — | — |
| 1-238 | 1000 | 30 | — | 40 | 0 | 0 | 0 | 0 | — | — | — |
| 1-242 | 1000 | 80 | 80 | — | — | 10 | 10 | 70 | — | 10 | — |
| 1-244 | 1000 | 90 | 90 | — | — | 60 | 60 | 70 | — | 70 | — |
| 1-252 | 1000 | 20 | — | 90 | 80 | 0 | 10 | 70 | — | — | — |
| 1-261 | 1000 | 70 | 70 | | | 0 | 20 | 40 | | 30 | |
| 1-262 | 1000 | 90 | — | 100 | 80 | 0 | 20 | 70 | — | — | — |
| 1-298 | 1000 | 10 | — | 0 | 10 | — | 20 | 10 | — | — | — |
| 1-306 | 1000 | 70 | — | 0 | — | 0 | 10 | 60 | — | — | — |
| 1-314 | 1000 | 60 | — | 0 | 0 | 0 | 10 | 10 | — | — | — |
| 1-317 | 1000 | 100 | — | 100 | — | 60 | 60 | 90 | — | — | — |
| 1-326 | 1000 | 80 | — | — | — | 0 | — | 80 | — | 0 | 0 |
| 1-356 | 1000 | 40 | — | 70 | 60 | 0 | 10 | 70 | — | — | — |
| 1-359 | 1000 | 10 | — | 0 | 10 | — | 20 | 10 | — | — | — |
| 1-385 | 1000 | 90 | — | 100 | 90 | 50 | 40 | 70 | — | — | — |
| 1-389 | 1000 | 80 | — | 50 | — | 10 | 0 | 70 | — | — | — |
| 1-391 | 1000 | 70 | — | 50 | — | 30 | 10 | 80 | — | — | — |
| 1-395 | 1000 | 40 | — | 20 | 20 | 10 | 0 | 70 | — | — | — |
| 1-396 | 1000 | 10 | — | 0 | — | 0 | 10 | 60 | — | — | — |
| 1-397 | 1000 | 70 | — | — | — | 20 | — | 70 | 80 | 10 | 10 |
| 1-401 | 1000 | 70 | — | 80 | 80 | — | 10 | 80 | — | — | — |
| 1-403 | 1000 | 90 | — | 80 | 80 | — | 20 | 70 | — | — | — |
| 1-406 | 1000 | 80 | — | 70 | 70 | 20 | 40 | 80 | — | — | — |
| 1-407 | 1000 | 70 | — | 30 | — | 0 | 0 | 80 | — | — | — |
| 1-409 | 1000 | 70 | — | 40 | — | 10 | 10 | 80 | — | — | — |
| 1-416 | 1000 | 80 | — | 90 | — | 10 | 20 | 80 | — | — | — |
| 1-418 | 1000 | 50 | — | — | — | 0 | — | 40 | 90 | 0 | 0 |
| 1-420 | 1000 | 80 | 70 | — | — | 10 | 10 | 70 | — | 10 | — |
| 1-428 | 1000 | 90 | 90 | — | — | 10 | 10 | 90 | — | 0 | — |
| 1-444 | 1000 | 70 | 60 | — | — | 10 | 10 | 50 | — | 20 | — |
| 1-452 | 1000 | 80 | 80 | — | — | 50 | 40 | 70 | — | 50 | — |
| 1-458 | 1000 | 90 | 90 | — | — | 90 | 40 | 70 | — | 90 | — |
| 1-459 | 1000 | 80 | 80 | — | — | 40 | 20 | 90 | — | 60 | — |
| 1-460 | 1000 | 90 | 90 | — | — | 20 | 10 | 60 | — | 40 | — |
| 1-461 | 1000 | 80 | 80 | — | — | 10 | 0 | 20 | — | 30 | — |
| 1-462 | 1000 | 80 | 80 | — | — | 0 | 10 | 70 | — | 20 | — |
| 1-463 | 1000 | 80 | 70 | — | — | 10 | 10 | 70 | — | 30 | — |
| 1-464 | 250 | 80 | 80 | — | — | 70 | 10 | 60 | — | 70 | — |
| 1-465 | 1000 | 80 | 80 | | | 60 | 20 | 70 | | 30 | |
| 2-65 | 1000 | 70 | — | — | — | 40 | — | 70 | 30 | 10 | 0 |
| 2-326 | 1000 | 70 | — | — | — | 0 | — | 40 | — | 0 | 0 |
| 2-418 | 1000 | 70 | — | — | — | 0 | — | 80 | — | 0 | 0 |
| 3-67 | 1000 | 50 | — | 0 | 10 | — | 20 | 50 | — | — | — |
| 3-297 | 1000 | 10 | — | 0 | 10 | — | 20 | 10 | — | — | — |
| 3-397 | 1000 | 10 | — | 100 | 90 | 0 | 0 | 60 | — | — | — |
| 3-419 | 1000 | 20 | — | 100 | 80 | 0 | 0 | 70 | — | — | — |
| 5-80 | 1000 | 20 | — | 10 | — | 0 | 10 | 10 | — | — | — |
| 5-251 | 1000 | 40 | — | 20 | 20 | 0 | 0 | 20 | — | — | — |
| 7-31 | 1000 | 0 | — | 0 | 0 | — | 20 | 0 | — | — | — |
| 7-75 | 1000 | 70 | — | 90 | — | 60 | 10 | 20 | — | — | — |
| 7-231 | 1000 | 90 | — | 100 | 90 | 10 | 30 | 80 | — | — | — |
| 7-237 | 1000 | 50 | — | 0 | 0 | 10 | 0 | 60 | — | — | — |
| 7-250 | 1000 | 100 | — | 100 | 90 | 30 | 20 | 80 | — | — | — |
| 7-253 | 1000 | 60 | — | 60 | 40 | 0 | 10 | 70 | — | — | — |
| 7-254 | 1000 | 50 | — | 60 | 30 | 0 | 20 | 30 | — | — | — |
| 7-325 | 1000 | 60 | — | 60 | 50 | — | 20 | 50 | — | — | — |

1. Use of a compound of formula (I), 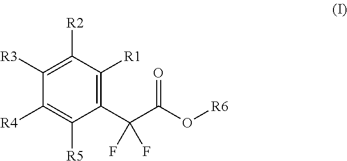
wherein:
each R1, R2, R4, and R5 is independently selected from the group consisting of hydrogen, halogen, amino, cyano, nitro, hydroxyl, C₁-C₅ alkyl, C₃-C₆cycloalkyl, C₁-C₄ alkoxy, C₂-C₃ alkenyl, C₂-C₃ alkynyl, C₁-C₂ haloalkoxy, halophenyl, C₁₋₂ haloalkyl, C₁₋₂alkoxyC₁₋₂alkyl, C₁₋₂ alkoxycarbonyl, C₁-C₂ alkylsulfanyl, C₁-C₂ alkylsulphinyl and C₁-C₂ alkylsulfonyl, wherein no more than one of R1, R2, R4, and R5 is C₁-C₄alkoxy, or
R1 and R2 together with the carbon atoms to which they are attached form a 5- or 6-membered ring, wherein the 6-membered ring contains zero, one or two nitrogen atoms with the proviso that any nitrogen in the 6-membered ring is next to the benzene ring in structure (I), and wherein the 5-membered ring contains one or two heteroatoms independently selected from the group consisting of nitrogen and oxygen, and each R4, and R5 is independently selected from the group consisting of hydrogen, halogen and C₁-C₂alkyl;
R3 is selected from the group consisting of hydrogen, halogen, amino, cyano, hydroxyl, C₁-C₅alkyl, C₁-C₄alkoxy, C₂-C₃alkenyl, C₂-C₃alkynyl, C₁-C₂haloalkoxy and halophenyl; at least one of R1, R2, R3, R4, and R5 is selected from the group consisting of amino, cyano, nitro, hydroxyl, C₂-C₅alkyl, C₃-C₆cycloalkyl, C₁-C₄alkoxy, C₂-C₃alkenyl, C₂-C₃alkynyl, C₁-C₂haloalkoxy, halophenyl, C₂haloalkyl, C₁₋₂alkoxyC₁₋₂alkyl, C₁₋₂ alkoxycarbonyl, C₁-C₂ alkylsulfanyl, C₁-C₂ alkylsulphinyl and C₁-C₂alkylsulfonyl, or R1 and R2 together with the carbon atoms to which they are attached form a 5- or 6-membered ring, wherein the 6-membered ring contains zero, one or two nitrogen atoms with the proviso that any nitrogen in the 6-membered ring is next to the benzene ring in structure (I), and wherein the 5-membered ring contains one or two heteroatoms independently selected from the group consisting of nitrogen and oxygen, and each R4, and R5 is independently selected from the group consisting of hydrogen, halogen and C₁-C₂alkyl; and
R6 is selected from the group consisting of hydrogen, benzyl and C₁-C₃alkyl;
or an agronomically acceptable salt of said compound, as a herbicide.

2. Use according to claim 1, wherein in the compound of Formula (I) R6 is hydrogen.

3. Use according to claim 1, wherein in the compound of Formula (I) R3 is hydrogen.

4. Use according to claim 1, wherein in the compound of Formula (I) each of R4 and R5 is selected from a group consisting of hydrogen and halogen.

5. Use according to claim 1, wherein in the compound of Formula (I) each R1, R2, R4, and R5 is independently selected from the group consisting of hydrogen, halogen, cyano, nitro, C₁-C₂alkylsulfanyl, C₁-C₄alkoxy and C₁-C₃alkyl.

6. Use according to claim 1, wherein in the compound of Formula (I) each R1 and R2 is independently selected from the group consisting of halogen, cyano, nitro, C₁-C₃alkyl, and C₁-C₂alkoxy, or
R1 and R2 together with the carbon atoms to which they are attached form a 5- or 6-membered ring, wherein the 6-membered ring contains zero, one or two nitrogen atoms with the proviso that any nitrogen in the 6-membered ring is next to the benzene ring in structure (I), and wherein the 5-membered ring contains one or two heteroatoms independently selected from the group consisting of nitrogen and oxygen, and each R4, and R5 is independently selected from the group consisting of hydrogen, halogen and C1-C2 alkyl.

7. Use according to claim 1, wherein in the compound of Formula (I) one of R1 and R2 is C₁-C₄alkoxy and the other of R1 and R2 is hydrogen.

8. Use according to claim 1, wherein in the compound of Formula (I) R1 and R2 together with the carbon atoms to which they are attached form a 5-membered heterocylic ring, wherein the 5-membered heterocylic ring is fully or partially saturated.

9. Use according to claim 1, wherein in the compound of Formula (I) R1 and R2 together with the carbon atoms to which they are attached form a 6-membered ring, wherein the 6-membered ring is aromatic.

10. Use according to claim 1, wherein in the compound of Formula (I) R1 and R2 together with the carbon atoms to which they are attached form a 5- or 6-membered ring, wherein the 5- or 6-membered ring is substituted by one to three substituents independently selected from the group consisting of halogen, C₁-C₂alkyl and C₁₋₃alkoxycarbonylC₁₋₃alkyl.

11. Use according to claim 1, wherein in the compound of Formula (I) at least one of the one to three substituents is C₁-C₂alkyl or C₁₋₃alkoxycarbonylC₁₋₃alkyl and it is substituted by one or two halogen.

12. A herbicidal composition comprising (i) a compound of Formula (I) or an agronomically acceptable salt of said compound as defined in claim 1 and (ii) an agriculturally acceptable formulation adjuvant.

13. A herbicidal composition according to claim 12, further comprising at least one additional pesticide.

14. A herbicidal composition according to claim 13, wherein the additional pesticide is a herbicide or herbicide safener.

15. A method of controlling weeds at a locus comprising application to the locus of a weed controlling amount of a compound of Formula (I) or an agronomically acceptable salt of said compound as defined in claim 1.
